(19) United States
(12) Patent Application Publication
(10) Pub. No.: US 2026/0283674 A1
(43) Pub. Date: Sep. 24, 2026

(54) CRYOGENIC CATHETER, SYSTEM, AND METHOD FOR SELECTIVE ABLATION OF MUCOSA AND SUBMUCOSA OF THE GASTROINTESTINAL TRACT

(71) Applicant: AGIL THERAPEUTICS, INC., San Francisco, CA (US)

(72) Inventors: Peter GARCIA-MEZA, San Francisco, CA (US); Timothy D HOLLAND, Los Gatos, CA (US)

(21) Appl. No.: 19/136,714

(22) PCT Filed: Dec. 6, 2023
(86) PCT No.: PCT/US2023/082782

§ 371 Date: Jun. 6, 2025

Related U.S. Application Data
(60) Provisional application No. 63/430,656, filed on Dec. 6, 2022.

(51) Int. Cl. A61B 18/02; A61B 18/00; A61M 25/00

(52) U.S. Cl. CPC A61B 18/0218; A61B 2018/00482; A61B 2018/00577; A61B 2018/00744; A61B 2018/0212; A61M 2025/0036

(57) ABSTRACT

Disclosed is a cryogenic catheter including a probe at the distal end, a shaft at the proximal end, and a cryogenic fluid delivery channel assembly operably coupled to the probe and shaft. The cryogenic fluid delivery channel assembly includes a cryogenic sprayer component, a catheter support wire, and a catheter overtube. The cryogenic sprayer component includes at least one sprayer tube and at least one delivery channel. The sprayer tubes include at least one sprayer through which a cryogenic fluid transported within the at least one delivery channel can be delivered to one or more target areas or treatment zones. The cryogenic fluid delivery channel assembly does not require the use of a central rail. The cryogenic catheter can be used in cryogenic ablation systems and methods involving cryogenic ablation of mucosal tissue and/or of both mucosal tissue and submucosal tissue in the gastrointestinal tract of a subject.

### CROSS-REFERENCE TO RELATED APPLICATIONS

This application is a U.S. National Phase filing under 35 U.S.C. § 371 of International Application No. PCT/US2023/082782, filed Dec. 6, 2023, and published as WO 2024/123945 A2 on Jun. 13, 2024, which claims priority benefit of U.S. Provisional Patent Application No. 63/430,656, filed Dec. 6, 2022, the disclosures of which are hereby incorporated by reference herein in their entirety.

### FIELD OF THE DISCLOSURE

The present disclosure generally relates to, inter alia, catheters, catheter probes, systems, and methods of using the catheters, catheter probes, and systems for cryogenic ablation of tissue, particularly of the gastrointestinal tract.

### BACKGROUND OF THE DISCLOSURE

The use of freezing for the ablation of unwanted tissue in medical applications has been used since the 1800's. The application of freezing temperature to tissue or cryoablation forms intracellular ice crystals which result in damage to cellular organelles and plasma membrane. Additionally, the free water is drawn during the freeze process, resulting in tonicity increase of the extracellular space. The increased solute concentration draws out the intracellular water, resulting in damage to cytoplasmic proteins and the destabilization of the cellular membrane. Although the exact mechanism of cell death is not known in cryoablation, the primary thought is that the disruption to the organelle membrane (i.e., mitochondria) and cellular plasma membrane initiates the cell death process. In particular, ice formation increases mitochondrial membrane permeability, which leads to disruption of the electron transport chain and irreversible mitochondrial de-energization, resulting in cell death. Cryoablation has shown to leave the acellular structural scaffolding (extracellular matrix) intact. Maintaining the structure allows for improved long-term regeneration of the tissue.

In contrast, high-temperature based ablation (e.g., radiofrequency ablation (RFA), microwave ablation, high-intensity focused ultrasound, laser, steam, hot-balloon) causes a coagulative denaturation to both cellular and acellular tissue structures. Further damage to the acellular tissue structure may result in severe unintended damage to the treated tissue, such as esophageal perforation and atrial-esophageal fistula. Additionally, a more intense inflammatory response is often observed in heat-based methods, as compared to cryo-based procedures.

The inflammatory response due to cryoablation, particularly in the submucosa, signals for the clearing of damaged cellular debris and initiation of tissue remodeling. All the commonly employed ablation therapies, such as radiofrequency ablation (RFA), microwave ablation, high-intensity focused ultrasound, laser, steam, and hot-balloon operate on the principle of hyperthermia. Only cryoablation, which is a hypothermic modality, induces tissue damage by a freeze-thaw process. Further, cryoablation causes minimal disruption to the microvasculature and extracellular matrix (ECM) particularly in the submucosa allowing for remodeling of the tissue layers with minimal to no fibrotic scar formation. An intact ECM provides cells with a scaffold for cellular migration, proliferation, and differentiation for tissue renewal.

Type 2 diabetes is a chronic condition that affects how the body metabolizes glucose. The effects of diabetes are staggering (over 10% of the US population has diabetes) to include being at higher risk of cardiovascular disease, retinopathy/macular oedema/glaucoma, nephropathy, neuropathy, and other conditions (1). Conventional medical treatment of type 2 diabetes only partially achieves adequate glycemic control and a reduction in cardiovascular risk (2). Thus, other approaches are needed.

Although developed as a weight reduction therapy, gastric-bypass surgery such as RYGB (Roux-en-Y bypass) has been reported to improve or eliminate type 2 diabetes in 70 to 80% of postoperative patients (3). The rapidity of the correction of glucose concentration in the blood and good glycemic regulation might be a result of the secondary alteration in incretin (hormonal) signals from the antrum, duodenum, and the proximal jejunum to the pancreatic islets (4). RYGB has shown to decrease or eliminate hormonal or neural signal(s) that normally would emanate from the stomach, pylorus, duodenum, or jejunum upon exposure to nutrient passage through the gastrointestinal tract. While effective, RYGB carries a substantial risk of surgical morbidity and mortality. Endoscopic interventions that combine the remarkable effectiveness of RYGB while minimizing associated risks would be a highly desirable addition to available treatments.

A technique that mimics RYGB is the DJBS (duodenal-jejunal bypass sleeve). Unlike RYGB, the DJBS (U.S. Pat. No. 7,819,836) is a fully reversible, nonsurgical procedure while still leveraging an important aspect of RYGB (eliminating nutrient exposure to the duodenum and jejunum). The clinical effects of improved glycemic control of the DJBL provide more evidence for the role of the duodenum in the interplay of obesity, metabolic syndrome, and type 2 diabetes. However, based on the rather high adverse event rate of the DJBL, this technique will not be likely adopted (5).

An additional nonsurgical avenue of investigation is ablation of the wall of one or more of the same organs of the gastrointestinal tract. Ablation could result in a similar absence of hormonal or neural signals as in bariatric surgery. Ablation of the duodenum is of particular interest given that the duodenum is recognized as a metabolic signaling center that seems to play a role in regulating insulin action and, therefore, insulin resistance (6).

One specific technique is hydrothermal ablation (U.S. Pat. No. 9,757,535) of the mucosa of the duodenum or DMR (duodenal mucosal resurfacing). The desired ablation is limited to the superficial intestinal mucosa leaving any deeper structures untouched. The goal with DMR is restoration to a normal mucosal interface in the duodenum (7). DMR has been found to be feasible and safe in that it elicited glycemic improvement in sub optimally controlled T2D patients using oral glucose-lowering medication (8). While showing promise, DMR has shown adverse events such as increased postoperative pain and usability shortcomings such as the need for fluoroscopy which could limit widespread adoption.

An additional ablative technique uses a laser to selectively target the submucosa of the duodenal wall and neural structures (nerves cells as ganglions, plexuses, axon, etc.) within. The proposed technique EGM (U.S. Pat. No. 10,575,904) or endoscopic glycemic management targets a large portion of the duodenum as nerves of the duodenum travel throughout the submucosal layer of the duodenum. The application of a laser does address usability shortcomings of hydrothermal resurfacing of the duodenal mucosa such as the ability for direct visualization during ablation (9).

Nonsurgical implants mimicking bariatric surgery have shown some level of glycemic control yet have unacceptable levels of adverse events. Promising ablative technologies are being introduced where glycemic improvements have been demonstrated and/or more clinical Investigation needs to be demonstrated. No single treatment has been demonstrated to be endoscopic and noninvasive that could target both the mucosa and the richly innervated submucosa which both play a role in resulting changes of hormonal and neural signals that are changed in bariatric surgery while at the same time not resulting in high rates of adverse events.

The present invention is directed to overcoming these and other deficiencies in the art.

### SUMMARY OF THE DISCLOSURE

The present disclosure generally relates to medical devices such as cryogenic catheters, cryogenic catheter probes, cryogenic ablation systems, and methods for their use for selectively ablating of the mucosa and submucosa in the gastrointestinal tract, including, inter alia, for the treatment of Type 2 Diabetes, obesity, and other metabolic and medical conditions.

In one aspect, the present disclosure provides a cryogenic catheter including a probe at the distal end of the catheter, a shaft at the proximal end of the catheter, and a cryogenic fluid delivery channel assembly operably coupled to and/or associated with the probe and shaft. The cryogenic fluid delivery channel assembly includes a cryogenic sprayer component, a catheter support wire, and a catheter overtube. The cryogenic sprayer component includes at least one sprayer tube and at least one delivery channel. The at least one sprayer tube includes at least one sprayer through which a cryogenic fluid transported within the at least one delivery channel can be delivered to one or more target areas or treatment zones.

In another aspect, the present disclosure provides a cryogenic ablation system. The cryogenic ablation system includes a cryogenic catheter as disclosed herein and a controller configured to control the functionality of the cryogenic catheter.

In another aspect, the present disclosure provides a method of performing cryogenic ablation of mucosal tissue and/or of both mucosal tissue and submucosal tissue in the gastrointestinal tract of a subject. This method includes the steps of: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In another aspect, the present disclosure provides a use of a cryogenic ablation system in a method of performing cryogenic ablation of mucosal tissue and/or of both mucosal tissue and submucosal tissue in the gastrointestinal tract of a subject. The method includes the steps of: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In another aspect, the present disclosure provides a cryogenic ablation system for use in a method of performing cryogenic ablation of mucosal tissue and/or of both mucosal tissue and submucosal tissue in the gastrointestinal tract of a subject, where the method includes the steps of: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In another aspect, the present disclosure provides a method for performing a medical procedure in a small intestine and/or stomach of a patient in need of the medical procedure. This method includes the steps of: (a) providing a cryogenic ablation system as disclosed herein; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In another aspect, the present disclosure provides a use of a cryogenic ablation system in a method for performing a medical procedure in a small intestine and/or stomach of a patient in need of the medical procedure. This method includes the steps of: (a) providing a cryogenic ablation system as disclosed herein; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In another aspect, the present disclosure provides a cryogenic ablation system for use in a method for performing a medical procedure in a small intestine and/or stomach of a patient in need of the medical procedure. This method includes the steps of: (a) providing a cryogenic ablation system as disclosed herein; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In certain aspects, the present disclosure relates to a cryogenic ablation system for treating tissue of the esophagus, stomach, duodenum, or jejunum which comprises a catheter and controller. The catheter is delivered to the gastrointestinal tract via a delivery channel of a standard endoscope, an accessory channel mounted to the side of a standard endoscope, or inside an over a tube or delivery sheath. All three methods allow the user to position the endoscope on the distal end of the catheter facilitating visualization through the distal end of the catheter.

In certain aspects, the catheter encompasses a probe, a catheter shaft, a handle, and a distal tip. The probe increases and decreases in diameter to conform within the gastrointestinal tract's anatomical passageways (e.g., esophagus, stomach, duodenum, or jejunum). When conformed within the passageway, the probe may be positioned at the desired location using the endoscope's visualization.

In certain aspects, the probe contains at least one cryogenic fluid delivery channel assembly. The probe's distal end is mounted to the distal probe tip and the catheter support wire runs through the distal probe tip, and the proximal end of the probe is mounted to the catheter shaft. The delivery channel(s) runs through a catheter overtube and are coupled together in the sprayer tube and slide along as a fixed unit.

In certain aspects, the controller encompasses a control system, a cryogenic fluid delivery system, a cryogenic supply system, and the controller connector. The control system contains hardware, software with associated algorithms, and a user interface, defining treatment parameters. The cryogenic fluid delivery system encompasses one or more control valves, one or more diverter valves, one or more motors, and one or more exhaust ports, which together with the control system automatically controls and directs the flow of cryogenic fluid at a predetermined time, a predetermined flow rate, to a selected cryogenic delivery channel(s), and cryogenic delivery channel(s) position, direction, translation speed. The cryogenic supply system encompasses one or more canisters that supply the cryogenic fluid for treatment. Once the high-pressure catheter connector is coupled to the controller connector, one or more cryogenic fluid delivery channels are fluidly coupled to the one or more canisters of cryogenic fluid.

In certain aspects, upon release from the controller and through one or more delivery channel(s), cryogenic fluid is directed outwardly toward the probe inner surface, causing the probe diameter to increase and come into intimate contact with the tissue of the gastrointestinal tract. The cryogenic ablation system is now ready for treatment, the present invention accurately controls probe inflation rate, allowing the probe to increase diameter for intimate contact with gastrointestinal tissue, probe inflation pressure, and probe temperature.

In certain aspects, upon delivery of the cryogenic fluid during treatment, cryogenic fluid makes contact with the inside of the probe wall causing cells in close proximity to the ablation interface (the interface of the probe wall and the contacted tissue or the inner layer of the mucosa) to undergo rapid freezing rates, whereas cells in the periphery of the ablation interface (submucosa and deeper layers of the wall of the small intestine) between the probe and the mucosa in intimate contact undergo moderate to lower freezing rates (10).

In certain aspects, the application of cryogenic fluid through the introduction into an expandable probe in intimate contact with the tissue of the gastrointestinal tract (mucosa) specifically of the small intestine leads to regenerated small intestine tissue. The effect is from the mucosa and into the submucosa where critical microvasculature in the submucosa is preserved (11). Temperature decrease to the submucosa facilitates modulation of the nerves of the submucosa (partial or reversible ablation, blocking, stimulation) while leaving critical microvascular structures intact which are necessary for regeneration. Further, the rapid freezing rates delivered to mucosal tissue modifies integrity and function of the intestinal barrier (mucosal epithelium) wherein the target mucosa intercellular spaces (ICS) decrease, and MI (mucosal impedance) increase thereby decreasing the permeability in mucosa ablated (12, 13).

In contrast to radiofrequency ablation, laser ablation, heated fluid ablation, and potentially other heat-based ablation techniques, cryoablation results in a robust inflammatory response, particularly in the submucosa. The inflammatory response creates the potential to stimulate additional responses particularly immunologic responses in cryoablation of tumors (14). Commonly employed ablation therapies in the clinical setting are radiofrequency ablation (RFA), microwave ablation, high-intensity focused ultrasound, and cryoablation. All these treatments operate on the principle of hyperthermia except for cryoablation, which is a hypothermic modality that induces tissue damage by a freeze-thaw process. Of all the ablation techniques, cryoablation demonstrated the highest potential to elicit a post-ablative immunogenic response (15). Finally, peptide bonds are not disrupted in the process of cryoablation so cold denaturation of proteins can be reversible with warming and rehydration (16).

In certain aspects, the present disclosure provides a cryogenic fluid delivery channel assembly for use in a cryogenic catheter probe, cryogenic ablation system, and in methods of use thereof for performing cryogenic ablation of mucosal tissue and/or of both mucosal tissue and submucosal tissue in the gastrointestinal tract of a subject. The cryogenic fluid delivery channel assembly does not require the use of a central rail. The cryogenic fluid delivery channel assembly can be adapted for use with one or more delivery channels for the cryogenic fluid. The one or more delivery channels can be arranged, bundled, and/or otherwise organized with the cryogenic fluid delivery channel assembly in various arrangements as disclosed herein. The one or more delivery channels can be combined at their ends with a sprayer for delivery of the cryogenic fluid in a desired pattern of distribution.

In one aspect, the present disclosure provides a cryogenic catheter probe including a chamber and a cryogenic fluid delivery channel assembly housed within the chamber. The chamber includes a distal end, a proximal end, and a hollow body portion disposed between the distal and proximal ends. The distal end of the chamber is adapted for connection with a distal probe tip. The proximal end of the chamber is adapted for connection with a catheter shaft. The cryogenic fluid delivery channel assembly: (i) a catheter support wire which is connected to the distal cap or the directly to the sprayer tube; (ii) a catheter overtube which is connected to the proximal cap or directly to the sprayer tube (iii) one or more delivery channels that transition into the sprayer tube and which each delivery channel is positioned independently or bound together allowing for translational positioning (iv) one or more sprayers which are in communication with the delivery channels tube and in fluid communication with the sprayers where the sprayer delivers cryogenic fluid to the probe wall inner circumference.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, if provided, like reference numerals contained in the drawings are meant to identify similar or identical elements.

FIG. 1 is a perspective view of the cryogenic ablation system and portions of the catheter and controller.

FIG. 2 is illustrative of the catheter exiting the endoscope within the duodenum positioned for treatment.

FIG. 3 is a front view of the controller with load cap(s), front panel, and display.

FIG. 4 is a back view of the controller and the mounting block.

FIG. 5 is an exploded perspective view of the controller.

FIG. 6 is an overview diagram demonstrating the organization of the control system.

FIG. 7 is a front of the controller with the front panel missing for better visualization of the internal components.

FIG. 8 is a schematic diagram of the cryogen path and phase.

FIG. 9 is an illustration of the wall of the duodenum.

FIG. 10 is a detailed cross-sectional image of duodenal tissue.

FIG. 11 is a detailed cross-sectional image of duodenal tissue, low magnification at 4 days post treatment.

FIG. 12 is a detailed cross-sectional image of duodenal tissue, high magnification at 4 days post treatment.

FIG. 13 is an illustration of the wall of the duodenum with emphasis on villi and crypts.

FIG. 14 is an illustration of the wall of the duodenum with emphasis on submucosa plexus and myenteric plexus.

FIG. 15 is a detailed cross-sectional image of duodenal tissue, low magnification at 4 days post treatment.

FIG. 16 is a detailed cross-sectional image of duodenal tissue, low magnification at 30 days post treatment.

FIG. 17 is a detailed cross-sectional image of duodenal tissue, high magnification at 4 days post treatment with emphasis on myenteric plexus between the inner and outer smooth muscle layers.

FIG. 18 is a detailed cross-sectional image of duodenal tissue, high magnification at 4 days post treatment with emphasis on submucosal plexus, adjacent to a cryoablation treatment site.

FIG. 19 is a cross-sectional side view of a portion of the catheter showing the cryogenic fluid delivery channel assembly.

FIG. 20 is a perspective view of the probe along with the cryogenic fluid delivery channel assembly.

FIG. 21 is a side view of the catheter in the load position. The controller and majority of probe omitted for clarity.

FIG. 22 is a side view of the catheter in the start treatment position. The controller and majority of the probe omitted for clarity.

FIG. 23 is a side view of the catheter in the complete treatment position. The controller and majority of the probe omitted for clarity.

FIG. 24 is a cross-sectional view of the cryogenic fluid delivery channel assembly.

FIG. 25 is a cross-sectional view of the cryogenic fluid delivery channel assembly and illustrates the treatment zone. Distribution of the cryogenic fluid from two of the three sprayers is shown, with the distribution covering two treatment zones.

FIG. 26 is a cross-sectional view of the cryogenic fluid delivery channel assembly and illustrates application of cryogenic fluid from a sprayer. Distribution of the cryogenic fluid from one of the three sprayers is shown.

FIG. 27 is a perspective view and close up the cryogenic fluid delivery channel assembly.

FIG. 28 is a perspective view of the cryogenic fluid delivery channel assembly where the delivery channels inside the catheter overtube are shown.

FIG. 29 is a side view of the cryogenic fluid delivery channel assembly.

FIG. 30 is a cross-sectional view of the catheter overtube with individual delivery channels within the catheter overtube.

FIG. 31 is a cross-sectional view of the catheter overtube with individual delivery channels within the catheter overtube where delivery channels are positioned or bound together.

FIG. 32 is a cross-sectional view of the catheter overtube where delivery channels are encased within the catheter overtube.

FIG. 33 is a cross-sectional view of the catheter overtube with individual delivery channels where delivery channels are non-circular shaped.

FIG. 34 is a cross-sectional view of the cryogenic fluid delivery channel assembly and illustrates application of cryogenic fluid from a sprayer.

FIG. 35 is a cross-sectional view of the cryogenic fluid delivery channel assembly and illustrates application of cryogenic fluid from a sprayer.

FIG. 36 is a cross-sectional side view of a portion of the catheter showing cryogenic fluid delivery channel assembly.

FIG. 37 is a perspective view of the probe along with the cryogenic fluid delivery channel assembly.

FIG. 38 is a perspective view of the cryogenic fluid delivery channel assembly.

FIG. 39 is a perspective view of the cryogenic fluid delivery channel assembly where the delivery channels inside the catheter overtube are shown.

FIG. 40 is a side view of the cryogenic fluid delivery channel assembly.

FIG. 41 is a cross-sectional view of the cryogenic fluid delivery channel assembly.

FIG. 42 is a cross-sectional view of the cryogenic fluid delivery channel assembly.

FIG. 43 is a cross-sectional view of the cryogenic fluid delivery channel assembly where the delivery channel is positioned toward the middle of the sprayer.

FIG. 44 is a cross-sectional view of the cryogenic fluid delivery channel assembly where the delivery channel is positioned toward the back of the sprayer.

FIG. 45 is a cross-sectional side view of a portion of the catheter showing the cryogenic fluid delivery channel assembly.

FIG. 46 is a perspective view of the probe along with the cryogenic fluid delivery channel assembly.

FIG. 47 is a perspective view and close up of the cryogenic fluid delivery channel assembly where the delivery channels inside the sprayer tube are shown.

FIG. 48 is a perspective view of the cryogenic fluid delivery channel assembly where the catheter overtube is separated from the sprayer tube.

FIG. 49 is a side view of the cryogenic fluid delivery channel assembly.

FIG. 50 is a perspective view and close up of the cryogenic fluid delivery channel assembly where the delivery channels are inside the sprayer overtube.

FIG. 51 is a cross-sectional view of a portion of the catheter overtube.

FIG. 52 is a cross-sectional view of a portion of the catheter overtube at the location of one of the sprayers.

### DETAILED DESCRIPTION OF THE DISCLOSURE

The present disclosure relates to methods, devices, and systems including a cryogenic catheter, cryogenic catheter probe, a cryogenic ablation system (e.g., including a cryogenic catheter and a controller) for the treatment of metabolic conditions including, but not limited to, Type 2 diabetes, obesity, hypertension, non-alcoholic fatty liver disease, acid reflux, Barrett's esophagus, etc., through efficiently ablating the luminal layers of the gastrointestinal tissue, including, for example, the esophagus, stomach, pylorus, duodenum, jejunum, etc. Structures affected by the various devices, systems, and methods of the present disclosure include the mucosa, submucosa, and/or muscularis layers. Other structures within the layers that may also be affected by the devices, systems, and methods of the present disclosure include, without limitation, vasculature and/or nerve tissue. For the devices, systems, and methods of the present disclosure, application of cryogen fluid through the introduction into a probe (including, without limitation, an expandable probe) in intimate contact with the mucosa of tissues such as the small intestine, stomach, and/or the esophagus leads to remodeling of the intestinal tissue. The effect is from the mucosa and into the submucosa where critical microvasculature in the submucosa is preserved.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements but is not limited to possessing only those one or more elements. Likewise, an element of a system, device, or apparatus that “comprises,” “has,” “includes” or “contains” one or more features possesses those one or more features but is not limited to possessing only those one or more features. Likewise, an element of a system, device, or apparatus that “comprises,” “has,” “includes,” or “contains” one or more features possesses those one or more elements but is not limited to keeping only those one or more attributes.

The terms “proximal” and “distal” are used herein regarding a clinician manipulating the controller portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those of ordinary skill in the art will recognize various equivalent variations on the description that follows. Unless otherwise stated, in this application, specified relationships, such as parallel to, aligned with, or in the same plane as, mean that the specified relationships are within limitations of manufacturing processes and manufacturing variations. When components are described as being coupled, connected, being in contact, or contacting one another, they need not be physically directly touching one another unless specifically described as such. Like elements in various embodiments are commonly referred to with like reference numerals.

Table A is a listing of various components, elements, aspects, or items shown in the as-filed Figures. The abbreviation “Ref. No.” refers to any reference numerals included or added to the Figures and/or the present disclosure corresponding to the listed component, element, aspect, or item.

TABLE A

| Ref. No. | Element, Aspect, or Item | FIGS. |
|---|---|---|
| | Catheter (Cryogenic Catheter Probe) | 1 |
| | Cryogenic Ablation System | 1 |
| | Handle | 1 |
| | Controller | 1, 3, 4, 6, 7 |
| | Controller Connector | 1, 6, 7 |
| | Cryogenic Catheter (Agil's Cryoablation) | 2 |
| | Duodenum | 2 |
| | Endoscope | 2 |
| | Stomach | 2 |
| | Load Cap | 3, 4, 6, 7 |
| | Display | 3, 6 |
| | Front Panel | 3, 6 |
| | Block Body | 4, 6 |
| | Mounting Block | 4, 6 |
| | Mounting Wheel | 4, 6 |
| | Pole Insertion Hole | 4 |
| | Control System | 6 |
| | Motherboard (backside of display) | 6 |
| | Cryogenic Fluid Delivery System | 6, 7 |
| | Cryogenic Supply System | 6, 7 |
| | Cartridge Container | 7 |
| | Catheter Locking Motor | 7 |
| | Cryogenic Supply Block | 7 |
| | Delivery Channel Flow Valve | 7 |
| | Exhaust Chamber | 7 |
| | Exhaust Valve | 7 |
| | Flow/Pressure PCBA | 7 |
| | High Pressure Connector | 7 |
| | Main Flow Valve | 7 |
| | Treatment Motor | 7 |
| | Treatment Slide | 7 |
| | Mucosa | 9, 10 |
| | Muscularis | 9, 10 |
| | Submucosa | 9, 10 |
| | Villi | 9, 14 |
| | Crypt | 14 |
| | Myenteric Plexus | 14 |
| | Submucosal Plexus | 14 |
| | Cryogenic Fluid Delivery Channel Assembly | 19, 20, 21, 22, 23, 24, 26, 27, 28, |
| | | 29, 34, 35, 36, 37, 38, 39, 40, 41, |
| | | 42, 43, 44, 45, 46, 47, 49 |
| | Catheter Shaft | 19, 20, 21, 22, 23, 24, 27, 36, 38, |
| | | 39, 45, 46, 47, 48, 50 |
| | Probe | 19, 20, 21, 22, 23, 36, 37, 39, 45, |
| | | 46 |
| | Distal Probe Tip | 19, 20, 21, 22, 23, 36, 37, 45, 46 |
| | Catheter Overtube | 19, 20, 21, 22, 27, 28, 29, 30, 31, |
| | | 32, 33, 36, 37, 38, 40, 45, 46, 47, |
| | | 48, 49, 50, 51, 52 |
| | Catheter Support Wire | 19, 20, 22, 23, 28, 29, 36, 37, 39, |
| | | 40, 45, 46, 48, 49, 50 |
| | Sprayer | 19, 22, 24, 25, 26, 27, 28, 29, 34, |
| | | 35, 36, 38, 39, 40, 41, 42, 43, 44, |
| | | 45, 47, 48, 49, 50, 52 |
| | Sprayer Tube | 19, 26, 27, 28, 29, 34, 35, 36, 38, |
| | | 39, 41, 42, 43, 44, 45, 47, 48, 49 |
| | Distal Cap | 19, 29, 36 |
| | Proximal Cap | 19, 29, 36, 40 |
| | Catheter Support Wire | 20 |
| | Load Position | 21 |
| | Start Treatment Position | 22 |
| | Complete Treatment Position | 23 |
| | Pressure Detection Tube | 24 |
| | Treatment Zone | 25 |
| | Probe Inner Surface | 24, 25 |
| | Probe Wall | 24, 25 |
| | Delivery Channel | 24, 26, 27, 28, 29, 30, 31, 32, 33, |
| | | 34, 35, 38, 39, 40, 41, 42, 43, 44, |
| | | 47, 49, 51, 52 |
| | Cryogenic Fluid | 25, 26, 29, 34, 35, 40, 49 |

Various aspects of the present disclosure are also addressed by the following Paragraphs 1-38 and in the noted combinations thereof, as follows:
Paragraph 1: A cryogenic catheter comprising: a probe at the distal end of the catheter; a shaft at the proximal end of the catheter; and a cryogenic fluid delivery channel assembly coupled to the probe and shaft, said cryogenic fluid delivery channel assembly comprising a cryogenic sprayer component, a catheter support wire, and a catheter overtube, wherein the cryogenic sprayer component comprises at least one sprayer tube and at least one delivery channel, wherein the at least one sprayer tube comprises at least one sprayer through which a cryogenic fluid transported within the at least one delivery channel can be delivered to one or more target areas or treatment zones.
Paragraph 2: The catheter according to Paragraph 1, wherein the sprayer tube is securely fastened between a distal cap and a proximal cap distal cap.
Paragraph 3: The catheter according to Paragraph 2, wherein the cryogenic sprayer component comprises multiple sprayer tubes each containing a single cryogenic fluid delivery channel.
Paragraph 4: The catheter according to Paragraph 2, wherein the cryogenic sprayer component comprises a single sprayer tube containing multiple cryogenic fluid delivery channels.
Paragraph 5: The catheter according to Paragraph 1, wherein the cryogenic sprayer component comprises a single sprayer tube containing one or more cryogenic fluid delivery channels.
Paragraph 6: The catheter according to Paragraph 1, wherein the at least one sprayer comprises multiple sprayers radially aligned, radially staggered, or both radially aligned and staggered in reference to their positions on the sprayer tube.
Paragraph 7: The catheter according to Paragraph 1, wherein the cryogenic fluid delivery channel assembly is configured for translational movement longitudinally between the distal and proximal ends of the catheter.
Paragraph 8: The catheter according to Paragraph 7, wherein the translational movement occurs without requiring a central rail component.
Paragraph 9: The catheter according to Paragraph 1, further comprising a distal probe tip attached to the distal end of the probe.
Paragraph 10: The catheter according to Paragraph 1, wherein the cryogenic fluid delivery channel assembly comprises 1 to 20 delivery channels.
Paragraph 11: The catheter according to Paragraph 1, wherein the cryogenic fluid delivery channel assembly comprises a number of delivery channels selected from the group consisting of 1, 2, 3, 4, 5, 6, 7, and 8 cryogenic fluid delivery channels.
Paragraph 12: The catheter according to Paragraph 1, wherein the sprayers are configured to release the cryogenic fluid either in unison or independently of one another.
Paragraph 13: The catheter according to Paragraph 1, wherein each sprayer is configured to release the cryogenic fluid in a different treatment zone.
Paragraph 14: The catheter according to Paragraph 1, wherein the catheter is configured for selective ablation in the mucosa and submucosa of a gastrointestinal tract of a subject.
Paragraph 15: The catheter according to Paragraph 1, wherein the delivery channels are independently controlled and turned to an on or off position.
Paragraph 16: The catheter according to Paragraph 1, wherein the treatment area or treatment zone comprises mucosal and/or submucosal tissue of the large intestine, small intestine, stomach, esophagus, rectum, and anus.
Paragraph 17: A cryogenic ablation system comprising: a cryogenic catheter according to any one of Paragraphs 1-16; and a controller configured to control the functionality of the cryogenic catheter.
Paragraph 18: The system according to Paragraph 17, wherein the shaft is connected to a handle.
Paragraph 19: The system according to Paragraph 17, wherein the system further comprises a pressure detection tube disposed within the cryogenic catheter probe or shaft.
Paragraph 20: The system according to Paragraph 17, wherein the at least one sprayer is connected to the outside of the distal end of the at least one delivery channel.
Paragraph 21: The system according to Paragraph 17, wherein each delivery channel is fluidly connected to a delivery channel control valve and a reservoir of cryogenic fluid whereby the delivery channel can be controlled by actuation of each delivery channel control valve.
Paragraph 22: The system according to Paragraph 17, wherein each delivery channel is associated with a sprayer which allows for partial restriction of the cryogenic fluid and a delivery channel control valve controls release of the cryogenic fluid from a cryogenic supply block into each delivery channel.
Paragraph 23: The system according to Paragraph 17, wherein the cryogenic catheter probe is placed into an expanded state upon release of cryogenic fluid into the inside of the cryogenic catheter probe.
Paragraph 24: The system according to Paragraph 17, wherein the controller independently controls deliver of cryogenic fluid to each delivery channel via control valves at the proximal end of each delivery channel, and/or wherein a reservoir system allows for large ablation areas up to 10 cm and beyond of tissue in a partial-circumferential or full-circumferential ablations.
Paragraph 25: The system according to Paragraph 24, wherein the controller comprises one or more variable controller parameters used to control the functional assembly.
Paragraph 26: The system according to Paragraph 24, wherein the system further comprises at least one sensor constructed and arranged to produce a sensor signal.
Paragraph 27: The system according to Paragraph 24, wherein the controller is configured to perform closed-loop energy delivery to the functional assembly based on the sensor signal.
Paragraph 28: A method of performing cryogenic ablation of mucosal tissue and/or of both mucosal tissue and submucosal tissue in the gastrointestinal tract of a subject, said method comprising: (a) providing a cryogenic ablation system according to Paragraph 17; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.
Paragraph 29: The method according to Paragraph 28, wherein the target treatment region comprises mucosal tissue and/or both mucosal and submucosal tissue of the large intestine, small intestine, stomach, esophagus, rectum, or anus of the subject.
Paragraph 30: The method according to Paragraph 29, wherein treating the target treatment region comprises performing a series of tissue ablation steps, each comprising ablation of an axial length of the large intestine, small intestine, stomach, esophagus, rectum, or anus of the subject, wherein each ablation step is optionally preceded by a tissue expansion step.
Paragraph 31: The method according to Paragraph 28, further comprises adjusting at least one variable controller parameter based on the sensor signal.
Paragraph 32: Use of a cryogenic ablation system in a method of performing cryogenic ablation of mucosal tissue and/or of both mucosal tissue and submucosal tissue in the gastrointestinal tract of a subject, wherein said method comprises: (a) providing a cryogenic ablation system according to Paragraph 17; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.
Paragraph 33: A cryogenic ablation system for use in a method of performing cryogenic ablation of mucosal tissue and/or of both mucosal tissue and submucosal tissue in the gastrointestinal tract of a subject, wherein said method comprises: (a) providing a cryogenic ablation system according to Paragraph 17; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.
Paragraph 34: A method for performing a medical procedure in a small intestine and/or stomach of a patient in need of said medical procedure, the method comprising: (a) providing a cryogenic ablation system according to Paragraph 17; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.
Paragraph 35: The method according to Paragraph 34, wherein treating the target treatment region comprises performing a series of tissue ablation steps, each comprising ablation of an axial length of the small intestine or stomach tissue, wherein each ablation step is optionally preceded by a tissue expansion step.
Paragraph 36: The method according to Paragraph 34, further comprises adjusting at least one variable controller parameter based on the sensor signal.
Paragraph 37: Use of a cryogenic ablation system in a method for performing a medical procedure in a small intestine and/or stomach of a patient in need of said medical procedure, the method comprising: (a) providing a cryogenic ablation system according to Paragraph 17; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.
Paragraph 38: A cryogenic ablation system for use in a method for performing a medical procedure in a small intestine and/or stomach of a patient in need of said medical procedure, the method comprising: (a) providing a cryogenic ablation system according to Paragraph 17; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In one aspect, the present disclosure provides a cryogenic catheter probe including a chamber and a cryogenic fluid delivery channel assembly housed within the chamber. The chamber includes a distal end, a proximal end, and a hollow body portion disposed between the distal and proximal ends. The distal end of the chamber is adapted for connection with a distal probe tip. The proximal end of the chamber is adapted for connection with a catheter shaft. The cryogenic fluid delivery channel assembly includes: (i) a catheter support wire which is connected to the distal cap or the directly to the sprayer tube; (ii) a catheter overtube which is connected to the proximal cap or directly to the sprayer tube (iii) one or more delivery channels that transition into the sprayer tube and which each delivery channel is positioned independently or bound together allowing for translational positioning (iv) one or more sprayers which are in communication with the delivery channels tube and in fluid communication with the sprayers.

In various embodiments, a distal probe tip attached to the distal end of the probe.

In various embodiments, the cryogenic fluid delivery channel assembly with catheter support wire on the distal end and the catheter overtube on the proximal end are configured to be guided thereby.

In various embodiments, the cryogenic fluid delivery channel assembly comprises a catheter support wire and a catheter overtube where one or more delivery channels run so the cryogenic fluid delivery channel assembly to be guided thereby. For example, the combination of the catheter overtube, catheter support wire, and how the sprayers, sprayer tube, and delivery channels are connected and bound such that there is no need for a central rail. In certain configurations, the sprayer tube can be an extruded tube with multiple delivery channels inside with simple sprayers notched into the sides of the sprayer tube.

In various embodiments, the cryogenic fluid delivery channel assembly is configured so that it prevents rotation of the assembly while translating. However, in certain embodiments, the cryogenic fluid delivery channel assembly can be configured to allow for rotation of the assembly while translating.

In various embodiments, the cryogenic fluid delivery channel assembly comprises 1 to 20 delivery channels.

In various embodiments, the cryogenic fluid delivery channel assembly comprises a number of delivery channels selected from the group consisting of 1, 2, 3, 4, 5, 6, 7, and 8 delivery channels.

In various embodiments, the sprayers of each of the delivery channels are configured to release cryogenic spray either in unison or independently of one another.

In various embodiments, each sprayer is configured to release cryogenic spray in a different treatment zone along the probe.

In various embodiments, the catheter is configured for selective ablation in the mucosa and submucosa of a gastrointestinal tract of a subject.

In various embodiments, the delivery channels are independently controlled and turned to an on or off position.

In various embodiments, the treatment tissue comprises mucosal and/or submucosal tissue of the large intestine, small intestine, stomach, esophagus, rectum, and anus.

In another aspect, the present disclosure provides a cryogenic ablation system. The cryogenic ablation system includes a cryogenic catheter probe as disclosed herein; a catheter portion functionally connected to the cryogenic catheter probe; and a controller configured to control the functionality of the cryogenic catheter probe.

In various embodiments, the system further comprises a catheter shaft connected to the proximal end of the probe and/or running through all or a portion of the probe. In various embodiments, the catheter shaft is connected to a handle.

In various embodiments, the system further comprises a pressure detection tube disposed within the cryogenic catheter probe or catheter shaft.

In various embodiments, the sprayer is connected to the outside of the distal end of the delivery channel.

In various embodiments, each delivery channel is fluidly connected to a delivery channel control valve and a reservoir of cryogenic fluid whereby cryogenic fluid delivery channel can be controlled by actuation of each delivery channel control valve. As used herein, the term “control valve” may also be referred to as a “flow valve” and the like.

In various embodiments, each fluid delivery channel has a sprayer which allows for partial restriction of cryogenic liquid and a delivery channel control valve controls release of cryogenic fluid from the cryogenic supply block into each cryogenic fluid delivery channel.

In various embodiments, the sprayers may be positioned axially within cryogenic fluid delivery channel assembly which incorporates the catheter support wire and the catheter overtube.

In various embodiments, the cryogenic catheter probe is placed into an expanded state upon release of cryogenic fluid into the inside of the cryogenic catheter probe.

In various embodiments, the controller independently controls deliver of cryogenic fluid to each delivery channel via control valves at the proximal end of each delivery channel, and/or wherein a reservoir system allows for large ablation areas up to 10 cm and beyond of tissue in a partial-circumferential or full-circumferential ablations.

In various embodiments, the controller comprises one or more variable controller parameters used to control the functional assembly.

In various embodiments, the system further comprises at least one sensor constructed and arranged to produce a sensor signal.

In various embodiments, the controller is configured to perform closed-loop energy delivery to the functional assembly based on the sensor signal.

In another aspect, the present disclosure provides a method of performing cryogenic ablation of mucosal tissue and/or of both mucosal tissue and submucosal tissue in the gastrointestinal tract of a subject. This method includes the steps of: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In various embodiments, the target treatment region comprises mucosal tissue and/or both mucosal and submucosal tissue of the large intestine, small intestine, stomach, esophagus, rectum, or anus of the subject.

In various embodiments, treating the target treatment region comprises performing a series of tissue ablation steps, each comprising ablation of an axial length of the large intestine, small intestine, stomach, esophagus, rectum, or anus of the subject, wherein each ablation step is optionally preceded by a tissue expansion step.

In various embodiments, the method further comprises adjusting at least one variable controller parameter based on the sensor signal.

In another aspect, the present disclosure provides a use of a cryogenic ablation system in a method of performing cryogenic ablation of mucosal tissue and/or of both mucosal tissue and submucosal tissue in the gastrointestinal tract of a subject, where the method comprises: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In another aspect, the present disclosure provides a cryogenic ablation system for use in a method of performing cryogenic ablation of mucosal tissue and/or of both mucosal tissue and submucosal tissue in the gastrointestinal tract of a subject, where the method comprises: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In another aspect, the present disclosure provides a method for performing a medical procedure in a small intestine and/or stomach of a patient in need of the medical procedure. This method includes the steps of: (a) providing a cryogenic ablation system as disclosed herein; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In various embodiments, treating the target treatment region comprises performing a series of tissue ablation steps, each comprising ablation of an axial length of the small intestine or stomach tissue, where each ablation step is optionally preceded by a tissue expansion step.

In various embodiments, the method further comprises adjusting at least one variable controller parameter based on the sensor signal.

In another aspect, the present disclosure provides a use of a cryogenic ablation system in a method for performing a medical procedure in a small intestine and/or stomach of a patient in need of the medical procedure. This method includes the steps of: (a) providing a cryogenic ablation system as disclosed herein; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In another aspect, the present disclosure provides a cryogenic ablation system for use in a method for performing a medical procedure in a small intestine and/or stomach of a patient in need of the medical procedure. This method includes the steps of: (a) providing a cryogenic ablation system as disclosed herein; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In another aspect, the present disclosure provides a method for performing a medical procedure in an intestine of a patient, the method comprising: (a) providing a catheter as disclosed and/or contemplated herein for insertion into the intestine or a system comprising the catheter and a controller, said catheter comprising: (i) proximal and distal portions; (ii) a probe mounted to the distal portion; and (iii) one or more cryogenic liquid delivery channels delivering cryogen to the inside of the probe; (b) introducing the catheter into the patient; and (c) treating target tissue with the probe in contact with the target tissue, wherein the target tissue comprises mucosal (and/or submucosal) tissue of the small intestine and treatment comprises ablating at least a portion of the mucosal and submucosal tissue of the small intestine, and wherein the medical procedure is configured to treat at least one of type 2 diabetes, non-alcoholic fatty liver disease (NAFLD) or non-alcoholic steatohepatitis (NASH).

In another aspect, the present disclosure provides a method for ablating (regenerating) the mucosa of the small intestine and alter, stimulate, or reduce neural activity in the submucosa of the small intestine of a subject, the method comprising: (a) providing a catheter for insertion into the intestine or a system comprising the catheter and a controller, said catheter comprising: (i) proximal and distal portions; (ii) a probe mounted to the distal portion; and (iii) one or more cryogenic fluid delivery channels delivering cryogen to the wall of the small intestine, at a selected power density (W/cm²), wherein the cryogenic fluid delivery channel can delivery cryogen that can elevate (or lower) tissue temperature from −25 C to −190 C; and (b) lowering a temperature of the target area using the cryogenic fluid delivery channels of the catheter, thereby ablating the mucosa of the intestine and delivers therapeutic energy to the submucosa to at least partially alter, stimulate, or reduce the neural activity or within the submucosal layer while maintaining functional activity of other layers of the surrounding target area.

In certain embodiments, the medical procedure is further configured to treat a disease or disorder selected from the group consisting of: Type 2 diabetes; Type 1 diabetes; “Double diabetes”; gestational diabetes; hyperglycemia; pre-diabetes; impaired glucose tolerance; insulin resistance; and combinations thereof.

In certain embodiments, treating target tissue modifies at least one of (1) nutrient absorption by the target tissue, (2) hormonal signaling from the target tissue, (3) secretions of the target tissue.

In certain embodiments, treating target tissue modifies integrity and function of the intestinal barrier (mucosal epithelium) wherein the target mucosa intercellular spaces (ICS) decrease, and MI (mucosal impedance) increase thereby decreasing the permeability in mucosa ablated.

In certain embodiments, the sensory nerves comprise at least one nerve that is activated by food passing through the duodenum; and/or wherein the sensory nerves comprise at least one nerve that transmits signals from at least one of mechano-sensors or chemoreceptors located within the duodenal wall.

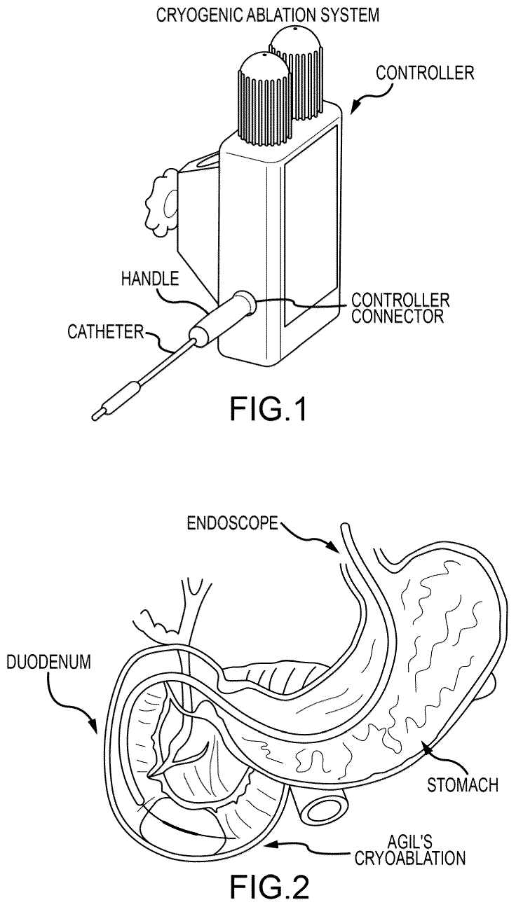

Referring now to FIG. 1, a schematic view of the cryogenic ablation system, consisting of a controller and a catheter. The catheter shown in FIG. 1 includes a catheter constructed according to the present invention's teachings and has a distal end and a proximal end. The proximal end of the catheter carries a connecting member through which the catheter is securely received into the controller. The catheter may be for a single-use, whereas the controller is reusable. The catheter is shown in the attached configuration with the controller.

Referring now to FIG. 2, an illustration of the embodiment of a cryogenic ablation system for performing a medical procedure on a patient. The cryogenic ablation system comprises a catheter and a controller. In this illustration, an embodiment of the catheter includes a probe which is a component of the catheter and is positioned and expanded for intimate contact with the targeted tissue as illustrated. The catheter is used in conjunction with an endoscope as illustrated. The distal end of such an endoscope in FIG. 2, has an imaging camera lens and illuminating light. The image is picked up by the lens is transferred via fiber optics to a monitoring camera, which sends TV signals via a cable to a conventional monitor, where the procedure can be visualized. By virtue of this visualization, the physician can perform cryoablation in the gastrointestinal tract. The catheter can be configured to be passed through the working channel of an endoscope. The probe is positioned on a distal portion of the catheter and is configured to be inflated by introducing cryogenic fluid into the catheter where the diameter of the probe may be increased or decreased to facilitate the introduction, removal, or positioning of the catheter and treatment using the catheter within the anatomical passageways.

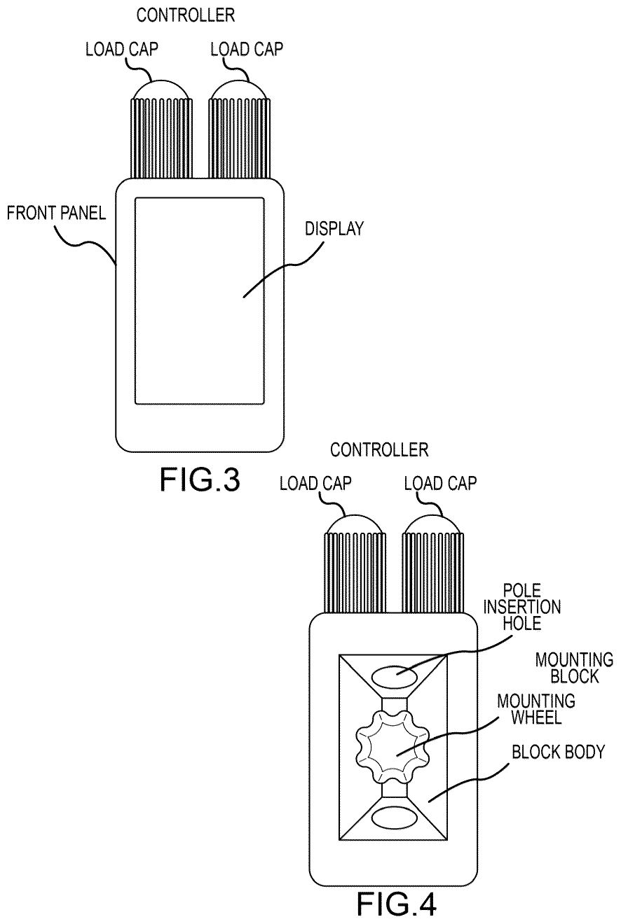

Referring to FIG. 3, which is a front view of the controller, comprises a display, load caps, and front panel. The display provides visual updates of the procedural, treatment, and informational status in real-time and allows user input and control while specifying details of the treatment. The controller may be configured with one or more cartridges that contain cryogenic fluid to accommodate different catheter configurations. The user will insert the canisters into the controller by unscrewing the load caps, inserting the cartridges into the controller, and tightening of load caps securely in place.

Referring now to FIG. 4 which is a back view of the controller, comprising load caps, mounting block, and the case. The mounting block includes pole insertion hole, block body, and mounting wheel. The controller has the means to be securely mounted to a pole such as an IV pole or other standard equipment stand. The user slides the rod into the pole insertion holes. Once at the appropriate position, the user tightens the mounting wheel to secure the controller in place.

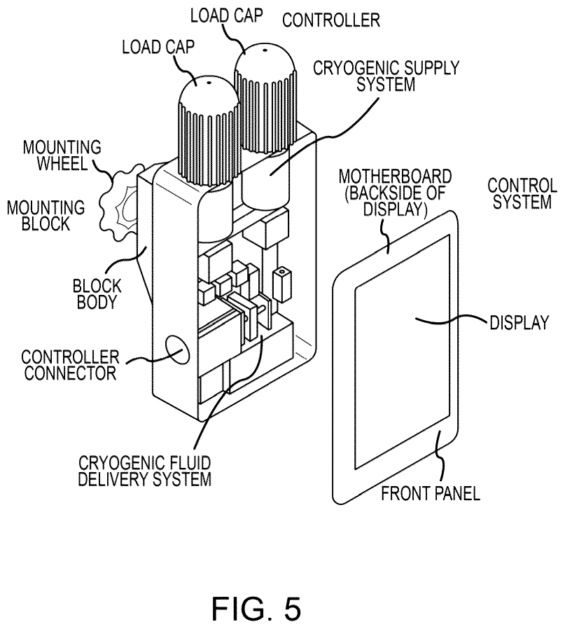

Referring to FIG. 5, which is a perspective view of the controller's embodiment, which encompasses a control system, a cryogenic fluid delivery system, a cryogenic supply system, and the catheter connector manifold with the front panel removed, exposing the internal components. The control system connects all electronic components, allowing them to communicate together. The control system encompasses the display, motherboard (on the backside of the front panel), hardware, software with associated algorithms, and user interface, which in combination define treatment parameters. The control system includes but is not limited to the CPU, memory, storage facility video, sound, and other ports.

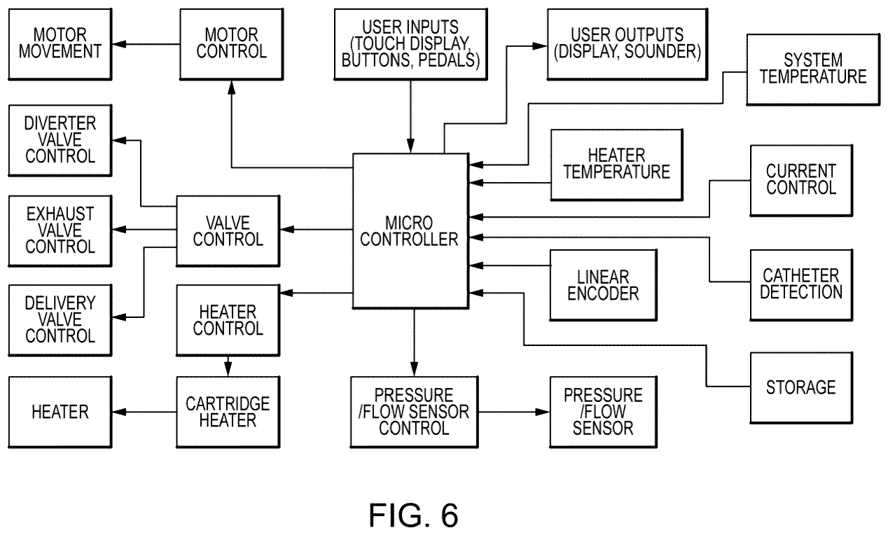

Referring to FIG. 6 is an overview diagram demonstrating the organization of the control system. The control system includes a display or user interface, a microcontroller, a motherboard, valve control, heater control, pressure and flow sensor control, motor control, user outputs, system temperature, catheter detection, storage, etc.

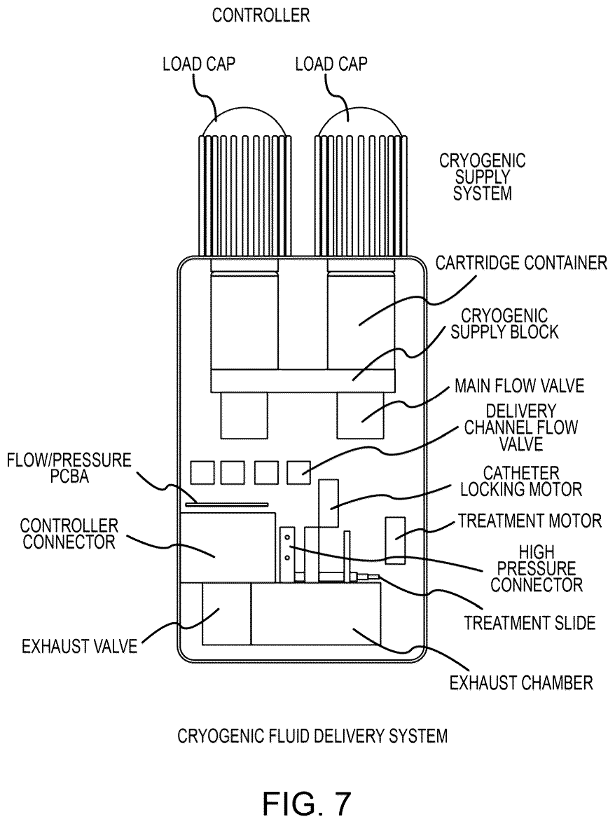

Referring to FIG. 7, an embodiment of the controller with the front cover missing for better visualization of the internal components. The controller's cryogenic supply system may include one or more load caps, one or more cartridges (residing within the load caps), one or more of the cartridge containers, and a cryogenic supply block that connects the cryogenic supply system to the cryogenic fluid delivery system. The cryogenic fluid delivery system consists of one or more main flow valves controlling the flow of cryogenic fluid from the cryogenic supply system into one or more delivery channel flow valve(s) which controls the flow of cryogenic fluid into one or more delivery channel(s). The control system controls the main flow valve(s) and delivery channel flow valve(s). The control system controls the cryogenic liquid delivery system to automatically direct the cryogenic fluid flow at a predetermined start and stop time by using an electronic or mechanical timer. The control system controls the linear traversing rate that is either distal or proximal by means of the treatment motor with an optical sensor or magnetic reader that translates the channels within the probe at a precise rate and location The controller connector aligns the appropriate ports from the high-pressure catheter connector, so the cryogenic supply system is fluidly coupled to the cryogenic fluid delivery channel(s). The cryogenic fluid delivery system encompasses the catheter locking motor, which keeps the catheter securely in place and connected with the controller at the catheter connector manifold. Within cryogenic fluid delivery system can be configured with liquid flow port, exhaust, and probe pressure port. Measurements from these ports may be inputs to a control algorithm implemented on the cryogenic liquid delivery system. The operation of the controller may be regulated or adjusted based on sensor feedback. In some embodiments, it may be desirable for the control algorithm to be fully automated, but the delivered therapy may utilize user input in other embodiments. The high-pressure catheter connector and the controller connector facilitate the liquid connection when both are securely locked together. Once securely locked together, the treatment motor traverses the cryogenic delivery channels within the probe's predefined configuration. The exhaust valve controls are fluidly coupled to the catheter and control the gas outlet from the probe and into the exhausting chamber. The controller can be configured to automatically control the outlet of gas from the probe through the exhaust valve before, during, and after the cryogenic fluid is flowing through the probe. Once the cryogen fluid has converted into a gas within the probe, it is conveyed back into the controller through the shaft inner space. It travels through the handle connector, exhaust valve, and into the exhaust chamber. Any remaining cryogen left within the cartridge after completing the therapy may be vented from the cartridge. The cartridge's venting conveys the cryogen fluid directly into the exhausting chamber. Once the cryogen has been contained within the exhaust chamber, it can be directed from the controller in several different ways. The exhaust from the system can be configured for direct or recovery of the gas.

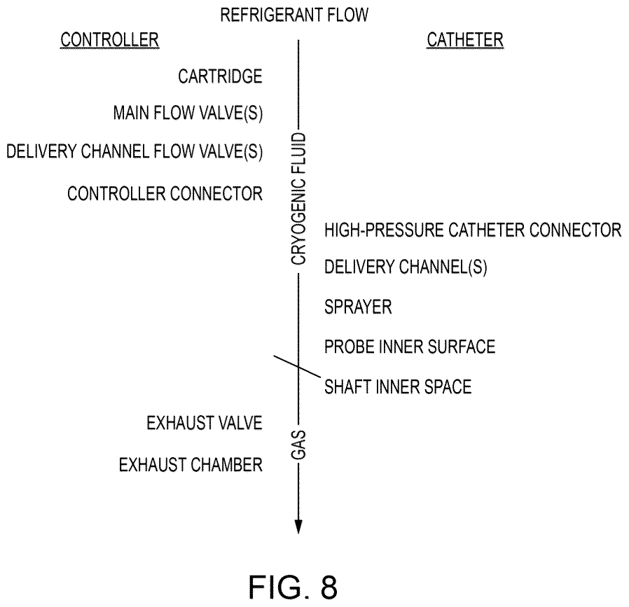

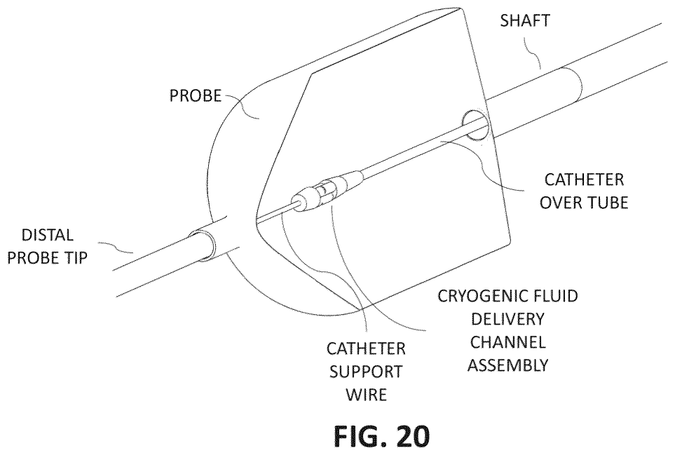

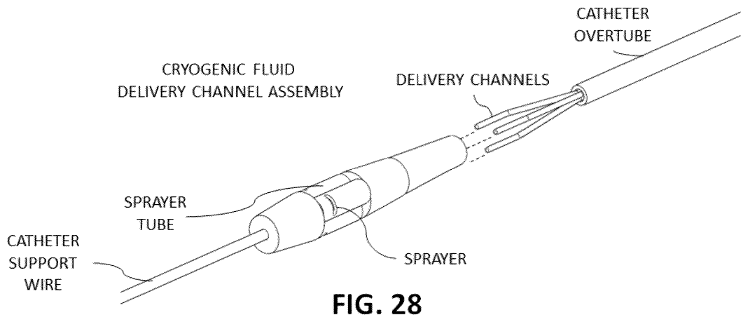

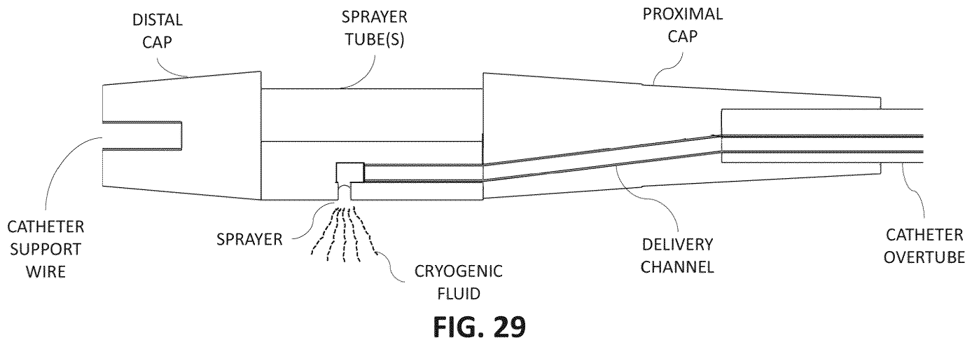

Referring to FIG. 8, an overview diagram illustrating the cryogenic pathway and phase of the cryogen (liquid, liquid+vapor or fluid, or gas phases) moves through the cryogenic ablation system components. In the canister, the cryogenic fluid is subcritical at room temperature, meaning that the fluid is both a liquid and vapor that coexist within the closed canister with the liquid being moderately dense liquid and where the cryogen is nitrous oxide or another cryogen such as CO₂. The cryogenic fluid is maintained within a specific pressure range that is not less than 250 psig and not greater than 1050 psig to ensure the right liquid density. Once the user has initiated the flow, the cryogenic fluid passes through the components within the controller such as the main flow valve(s) (FIG. 7), delivery channel flow valve(s) (FIG. 7), and the controller connector (FIG. 7). The cryogenic fluid flow continues through the catheter, which fluidly coupled to the controller, by flowing through the delivery channel(s) (FIG. 28.39), and the cryogenic fluid delivery channel assembly (FIGS. 20, 37, 46). Once the cryogenic fluid reaches the cryogenic fluid delivery channel assembly, the sprayers (FIGS. 29, 40, 49) are configured to direct the cryogen to the probe's inner surface and to the probe wall which is in intimate contact with the gastrointestinal tissue. This initiates an evaporative process where the cryogenic fluid is converted into a gas increasing in temperature resulting from the cryogenic fluid's liquid evaporation where heat has been absorbed from the probe wall which is in intimate contact with gastrointestinal tissue. The exhaust valve controls are fluidly coupled to the catheter and control the gas outlet from the probe, through the catheter shaft inner space and into the exhaust chamber. The controller can be configured to automatically control the gas outlet from the probe (FIG. 20) through the exhaust valve (FIG. 7) before, during, and after the cryogenic fluid is flowing through the probe. The gas is conveyed back into the controller through the catheter shaft inner space. The gas travels through the controller connector (FIG. 7), exhaust valve (FIG. 7), and into the exhaust chamber (FIG. 7). Any remaining cryogen left within the canister after completing treatment may be vented from the canister. The canister's venting conveys the cryogenic fluid directly into the exhaust chamber, where the fluid undergoes a phase change into gas. Once the gas has been contained within the exhaust chamber, it can be directed from the controller in several different ways. The exhaust from the system can be configured for direct or recovery of the gas.

The materials and methods of making the cryogenic catheter probe and cryogenic ablation system of the present disclosure are those materials and methods suitable for making catheters, controllers, catheter probes, and other related aspects as described herein and as described in the art.

### Uses of the Cryogenic Catheter and Cryogenic Ablation System of the Present Disclosure

The present disclosure relates to, inter alia, methods, devices, and systems including a catheter and controller for the treatment of metabolic conditions including and not limited to Type 2 diabetes, obesity, hypertension, non-alcoholic fatty liver disease, acid reflux, Barrett's esophagus, etc., through efficiently ablating the luminal layers of the esophagus, stomach, pylorus, duodenum, or jejunum. Structures affected by the invention includes the mucosa, submucosa, and/or muscularis layers. Other structures within the layers may also be affected by the invention include vasculature and/or nerve tissue. For this invention, application of cryogen fluid through the introduction into an expandable probe in intimate contact with the mucosa of the small intestine, stomach, and/or the esophagus leads to remodeling of the intestinal tissue. The effect is from the mucosa and into the submucosa where critical microvasculature in the submucosa is preserved.

Upon delivery of the cryogenic fluid during treatment, liquid cryogen makes contact with the inside of the probe wall causing cells in proximity to the ablation interface (the interface of the probe wall and the contacted tissue or the inner layer of the mucosa) to undergo rapid freezing rates, whereas cells in the periphery of the ablation interface (submucosa and deeper layers of the wall of the small intestine) between the probe and the mucosa in intimate contact undergo moderate to lower freezing rates.

Cryoablation has been successfully used to remove or reduce unwanted tissue with positive remodeling resulting in normal tissue. As an example, cryo has been found to be useful in the reduction of keloid scar lesion. Application of a freezing temperature will cause cellular death and reduction of unwanted tissue. During regeneration, the acellular extracellular matrix remains intact and allows for cellular regeneration to occur. This positive remodeling for treatment of metabolic conditions through cryoablation of the luminal layers of the esophagus, stomach, pylorus, duodenum, or jejunum may impact several mechanisms such as modifying enteroendocrine signaling, disrupting or remodulating afferent nerve terminals located in the duodenum, improving the mucosal barrier whereby improving absorption mechanisms of the stomach, duodenum, and jejunum, along with other mechanisms.

EECs (Enteroendocrine cells) in the mucosa play a key role in gut hormone signaling for regulating insulin, satiety, and gut movement. Ablating the mucosal layer of stomach, duodenal, and jejunum where enterocytes may rejuvenate and repopulate the mucosa (i.e., duodenal mucosa) with “healthier” EECs, resulting in improved regulation of glucose uptake and transportation. EECs localization is sparse and irregular in the mucosal epithelium. Proposed changes in mechanism of action may occur with the cryoablation of ECCs at the stomach, duodenum, and jejunum.

Cryoablation in the stomach, duodenum, and jejunum submucosal layer may potentially be therapeutic. The energy delivery can facilitate modulation of the submucosal plexus (partial or reversable ablation, blocking, stimulation) along with mild modulation to the myenteric plexus (partial or reversable ablation, blocking, stimulation), while leaving critical microvascular and structural proteins intact which are necessary for regeneration. Improved brain-gut signaling (chemo-receptors) may occur post cryoablation. Additionally, the effect to afferent nerve terminals at the duodenum would produce similar effect as disruption of the CCK, GIP

Upon duodenal-jejunal bypass surgery, epithelial proliferation and tight junction expression are increased, which subsequently leads to decreased intestinal permeability. In line with this observation, RYGB surgery is associated with decreased endotoxemia in patients with obesity and T2DM. The rapid freezing rates delivered to mucosal tissue modifies integrity and function of the intestinal barrier (mucosal epithelium) wherein the target mucosa intercellular spaces (ICS) decrease, and MI (mucosal impedance) increase thereby decreasing the permeability in mucosa ablated.

### The Wall of the Small Intestine:

The main functions of the small intestine are digestion, absorption of food and the production of gastrointestinal hormones. The duodenal wall is made of four tissue layers that are consistent with the structure of the rest of the gastrointestinal tract including the esophagus, stomach, and jejunum.

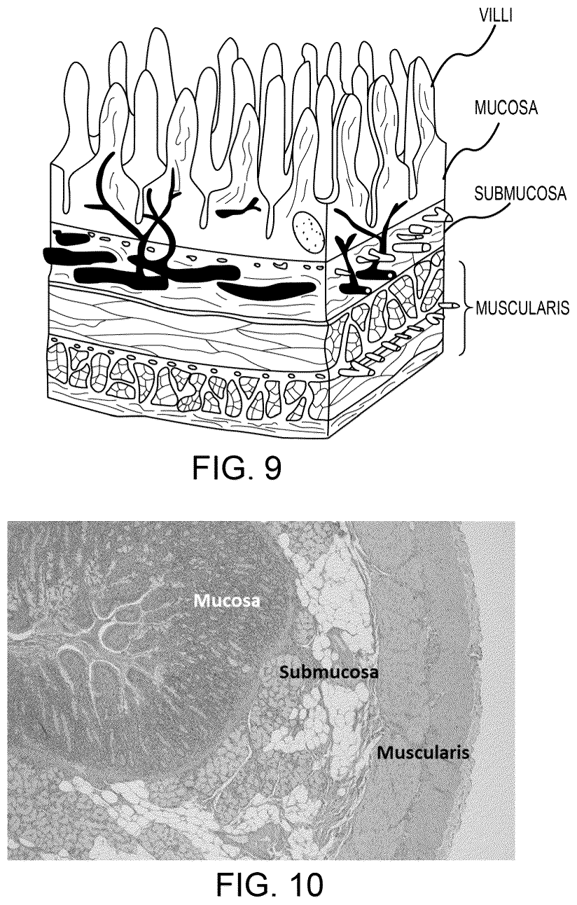

Referring to FIG. 9 and FIG. 10, the mucosa is the inner most layer which lines the inner surface of the duodenum and is in contact with chyme passing through the intestinal lumen. The mucosa is highly folded to increase surface area for nutrient absorption. Villi, which are smaller folds, are finger like mucosal projections. Columnar epithelial cells lining the surface have fine projections called microvilli which further increase the surface area and improve the absorption of nutrients. Plentiful mucus glands secrete mucus into the lumen to lubricate the intestinal wall and protect it from friction and acidic chyme (partially digested food).

Surrounding the mucosa layer is the submucosa, a layer of connective tissue that supports other tissue layers. Many blood vessels and nerve structures pass through the submucosa, specifically a network of nerve fibers and neuronal cell bodies called the submucosal plexus are present within the submucosa.

The muscularis layer surrounds the submucosa. This layer contains the smooth muscle tissue where the inner portion is a circular muscle layer, and the outer portion is a longitudinal layer of smooth muscle. The two layers of muscle in the muscularis layer sandwich the myenteric plexus that contains nerve fibers and parasympathetic ganglia.

Lastly, the serosa is the outermost layer of the duodenum that acts as the outer protective surface of the intestine. The serosa is comprised of loose connective tissue and a thin mesothelial cell layer, providing a smooth, slick surface to prevent friction between the duodenum and the surrounding organs.

### Mechanisms of Action of the Cryogenic Catheter and Cryogenic Ablation System of the Present Disclosure:

For this disclosure, application of cryogen fluid through the introduction into an expandable probe making intimate contact with the mucosa of the small intestine and/or the stomach leads to remodeling of the intestinal tissue. The effect is from the mucosa and into the submucosa where critical microvasculature in the submucosa is preserved. Remodeling of the mucosa is targeted at EECs which plays a key role in gut hormone signaling for regulation of insulin, satiety, and gut movement. Improper signaling by the EECs may lead to altered insulin production and/or function resulting in hyperglycemia. Additionally, remodeling of the duodenal mucosa relates to the disruption of the nerve terminals and plexus located in the mucosa.

Temperature decrease to the submucosa is therapeutic in the sense that the energy delivery facilitates modulation of the nerves of the submucosa (partial or reversable ablation, blocking, stimulation) while leaving critical microvascular and structural proteins intact which are necessary for regeneration. Additionally, cryoablation has been observed to not produce coagulative effect; thus, resulting in a lower incidence of thrombus formation as compared to RF and other high-temperature ablation technologies.

Further, the rapid freezing rates delivered to mucosal tissue modifies integrity and function of the intestinal barrier (mucosal epithelium) wherein the target mucosa intercellular spaces (ICS) decrease, and MI (mucosal impedance) increase thereby decreasing the permeability in mucosa ablated.

Cryoablation results in a robust inflammatory response, particularly in the submucosa. The inflammatory response signals for the clearing of damaged cellular debris and initiation of tissue remodeling. Commonly employed ablation therapies in the clinical setting are radiofrequency ablation (RFA), microwave ablation, high-intensity focused ultrasound, laser, steam, hot-balloon and cryoablation. All these treatments operate on the principle of hyperthermia except for cryoablation, which is a hypothermic modality that induces tissue damage by a freeze-thaw process.

Similar to microvasculature, cryoablation causes minimal disruption to the extracellular matrix (ECM) allowing for remodeling of the tissue layers with minimal to no fibrotic scar formation. An intact ECM provides cells with a scaffold for cellular migration, proliferation, and differentiation for tissue renewal.

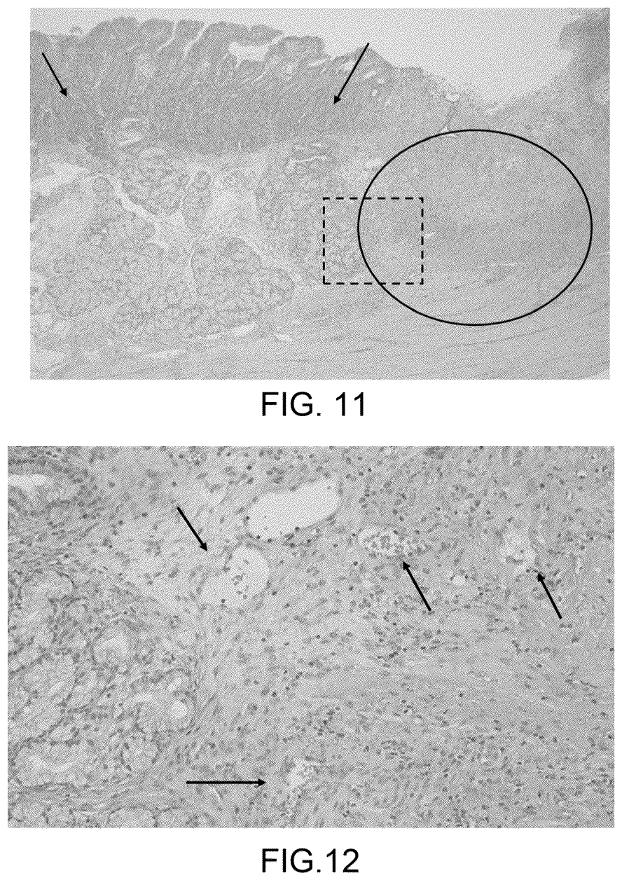

Referring to FIG. 11, a low magnification, H&E stain, represents a partial thickness section of the duodenum, at the edge of a cryoablation site at Day 4. The untreated intact mucosal epithelium is indicated by black arrows, and the ablation site, with loss of the mucosal epithelium and submucosal glands is the area within the black circle. Referring to FIG. 12. is a higher magnification of the black dashed box in FIG. 11, H&E stain, demonstrating viable blood vessels within the submucosa (black arrows). These blood vessels proliferate in conjunction with fibroblasts, forming granulation tissue at the ablation site associated with the healing response. Collagen bundles form part of the ECM associated with healing and tissue regeneration at the cryoablation site.

Cryoablation has been successfully used to remove or reduce unwanted tissue with positive remodeling of normal tissue. As an example, cryo has been found to be useful in the reduction of keloid scar lesion. Application of a freezing temperature will cause cellular death and reduction of unwanted tissue. During regeneration, the acellular extracellular matrix remains intact and allows for cellular regeneration to occur.

Cryoablation induces cell necrosis for therapeutic purposes through cycles of controlled local freezing and thawing of the tissue. Application of freezing temperatures to tissue result in necrosis of mucosal and submucosal layers by several mechanisms. Freezing of tissue results in the formation of ice crystals within the intracellular and extracellular spaces, leading to cell membrane disruption, protein denaturation, and osmotic gradients that lead to cell dehydration. This leads to necrosis in which intracellular contents (e.g., DNA, RNA and other intracellular contents) are released, and leading to an immune response. Cells in the periphery of ablation zones that are not immediately destroyed by direct cryoablation-induced injury may subsequently die by apoptosis, thought to be mediated by cytochrome C release. The thawing component of cryotherapy also appears to be an important mechanism for cell death. During thawing, ice crystals fuse and further damage cell membranes. In addition, vascular stasis due to endothelial damage, platelet aggregation, and formation of microthrombi results in ischemic necrosis. For cell destruction by cryotherapy, the tissue temperature must reach a critical threshold that is unique to the cell type and the environment of the targeted tissue, but typically ranges below freezing may be effective. Because collagen and elastin fibers are less sensitive to the effects of cryotherapy than are epithelial cells, the tissue structure remains intact, reducing the risk of perforation. The extent of tissue destruction is also dependent on the number of freeze/thaw cycles applied.

### Gastrointestinal Role in Enteroendocrine Regulation

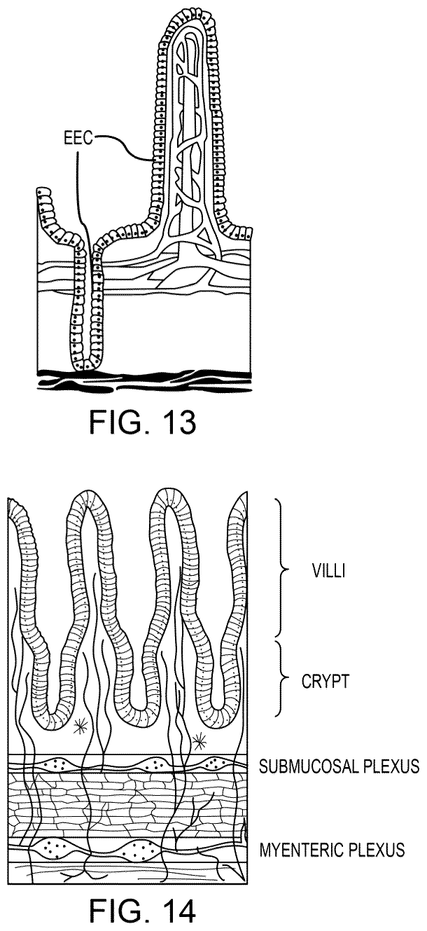

Enteroendocrine cells (EEC) are widely distributed throughout the gastrointestinal (GI) mucosa in crypts and villa, represent 1% of the total gut epithelium cell population, and form the largest endocrine organ in the body. Referring to FIG. 13 the illustration of an isolated villus and crypt. There is a wide distribution of EECs in the villi and crypts which are demonstrated and where the EECs are interspersed between non endocrine cells. EECs comprise different subgroups producing and releasing a variety of hormones under appropriate stimulation. EECs are largely distributed in the gut. In addition, Arteriole and Venule are shown along with a lymphatic vessel.

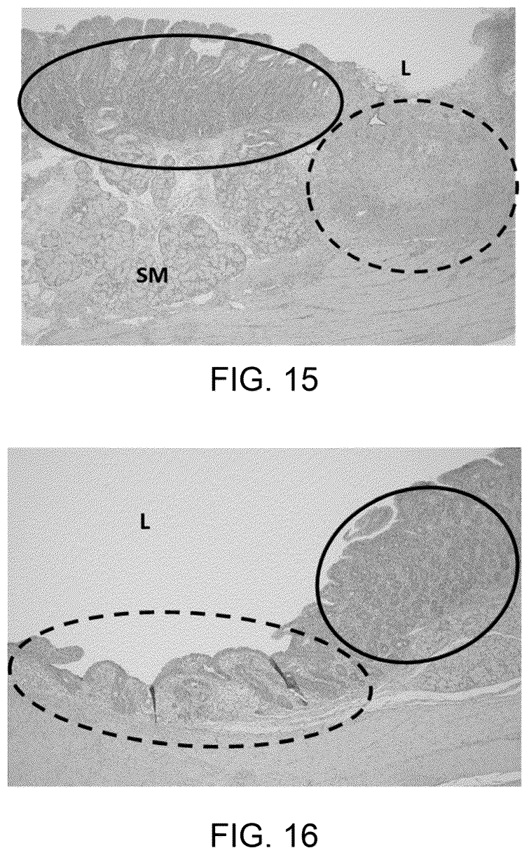

Referring to FIG. 14, the layers of the wall of the duodenum are represented. The anatomy of intestinal epithelium includes the mucosa depicting villi differentiated cell types and crypts. The submucosa containing submucosal plexus is depicted where intrinsic nerves from the submucosal plexus run from the submucosa into the villa in the mucosal layer. From the myenteric plexus, which is found between the circular muscle and the longitudinal muscle, run intrinsic nerves into the villa of the mucosa.

EECs plays a key role in gut hormone signaling for regulation of insulin, satiety, and gut movement. Improper signaling by the EEC may lead to altered insulin production and/or function resulting in hyperglycemia. Additionally, EECs are responsible for postprandial regulation of glucose level and metabolism. The EECs detects luminal content and release signaling molecules that can enter the circulation to act as classic hormones on distant targets, act locally on neighboring cells and on distinct neuronal pathways including enteric and extrinsic neurons.

EECs secrete more than 20 types of peptides/hormones. These secretory products can act locally in a paracrine manner, activating other EECs and other cell types in the mucosa, in addition, reach distant targets through release into the bloodstream or act directly on nerve endings close to the site of release. EEC secretory products are released in response to diverse types of stimuli and influence a variety of physiological functions. For example, GLP-1, glucagon like peptide-2 (GLP-2) and PYY, are contained in open type L cells and are released in response to ingested nutrients, including carbohydrates and fat.

Recent studies have shed light on EEC sensory transmission by showing direct connections between EECs and the nervous system via axon-like processes that form a well-defined neuroepithelial circuits through which EECs can directly communicate with the neurons innervating the GI tract to initiate appropriate functional responses.

In comparison to the distal gut, most ingested nutrients are absorbed within the proximal small intestine. The upper small intestine receives more vagal afferent innervation, which forms part of a neural circuit that mediates satiety; particularly, the duodenum is richly innervated by the parasympathetic nervous system which makes up the enteric nervous system (ENS). Sensory afferent neurons are the primary sensors and regulators that detect luminal contents. The afferent neurons transmit information to the brain, resulting in a gut response such as motility, intestinal barrier function, and epithelial secretion. Additionally, this system is a key regulator of insulin production and glycemic management. Dysfunction of the signaling pathway can lead to improper insulin production, leading to T2DM.

Sensory neurons, a subclass of enteric neurons, are the primary sensors and regulators of the ENS that detect luminal contents. These neurons respond to mechanical and chemical stimuli by activating intestinal muscles and controlling secretion of enzymes, hormones (by endocrine cells) and neurotransmitters. The ENS transmits information to the central autonomic nervous system through afferent nerves of the small intestine which correspond with specific areas in the brain (the well-known gut-brain axis). These areas are involved in metabolic regulation through controlling the function of splanchnic organs, such as the liver and endocrine pancreas, and the regulation of appetite and satiation. Interestingly, insulin has a direct regulatory effect on this pathway, resulting in the inhibition of food intake and weight control.

Additionally, it has been observed that a high-fat diet and T2D correlate with presence of neuropathy in the duodenal myenteric plexus, a decrease in the supporting enteric glial cells, and the loss of duodenal neurons. Recovery of these neuronal pathways can be achieved; it has been suggested that both RYGB and vertical sleeve gastrectomy lead to improved energy homeostasis and metabolism by manipulation of vagal afferent fibers of the ENS.

The vagal afferent innervation of the antral mucosa consists of fibers that pass through the submucosa to the level of the muscularis mucosa, where the neurites then arborize extensively.

Because these neuronal GLP-1 projections (see below) receive input from the gut via vagal afferents, likely including GLP-1 sensitive afferents from the intestinal mucosa and the hepato-portal vein, they might be considered as an additional mechanism for amplification of the rapidly fading peripheral GLP-1 signal.

### Cryomodulation of Enteroendocrine Signaling:

Targeted treatment of the EECs in the duodenum can improve glycemic regulation by altering endocrine signaling. This section provides details of the proposed changes to EEC signaling pathways with cryotreatment of the invention.

### A. Enteroendocrine Hormones and the Incretin Effect

There are various enteroendocrine hormones that have been observed to play a role in nutrient detection, motility, secretion, inflammatory response, satiety, and insulin regulation. Glucagon-like peptide 1 (GLP-1) and gastric inhibitory polypeptide (GIP) are key incretin hormones that are intimately involved in the regulation of glucose homeostasis. Endocrine L and K cells are located throughout the small intestine, and they secrete gut hormones in the presence of nutrients in the lumen. K cells are predominantly found in the duodenum and secretes GIP in response to luminal carbohydrate, dietary lipid and glucose. GLP-1 are contained in open type L cells which are found in the small intestine and the colon and are released in response to ingested nutrients, including carbohydrates and fat.

These incretin hormones ensure that postprandial glucose levels do not increase excessively. Referred to as the incretin effect, oral carbohydrate administration stimulates the secretion of GIP (glucose-dependent insulinotropic polypeptide) and GLP-1 which stimulate insulin secretion, and the stimulated insulin secretion, in turn, is responsible for the increased disposal of glucose.

It has been demonstrated that the incretin effect is severely reduced or even absent in patients with type 2 diabetes, even in patients with a considerable insulin secretory capacity. The incretin deficiency is therefore one of the most important pathogenetic factors behind the impaired glucose tolerance in this disease.

### B. Anti-Incretin Effect

Glucose regulation not only depends on the amount of GLP-1 and GIP being secreted, but also the number of active hormones available to promote insulin secretion. Both GLP-1 and GIP are rapidly degraded by the enzyme DPP-4 (Dipeptidyl peptidase-4), which is an anti-integrin broadly expressed on cell surfaces and present in the circulation. GLP-1 is rapidly cleaved by the DPP-4 into GLP-1 (9-36), which exhibits its own biological activities (albeit not insulin secretion anymore). GIP is also cleaved by DPP-4 into inactive GIP (3-42). As a result, the majority of GLP-1 and GIP is inactivated before reaching the systemic circulation; inhibiting the insulinogenic effect. DPP-4 inhibitors are a class of drugs commonly used to treat T2DM by reducing the inactivation of GLP-1, resulting in reduced levels of blood glucose.

### C. Cryoablation of Duodenum and Stomach

It is proposed that the cryotreatment of the duodenum and/or stomach may provide a similar effect as RYGB. In the short-term, nutrients that are not absorbed due to the ablation of the upper small intestine and stomach will primarily be absorbed in the lower portion. Disrupting the tissue at the upper small intestine and stomach may inhibit its response nutrient contact. This could lead to an exaggerated secretion of GLP-1 and increased signaling for insulin secretion. Further, ablation of K-cells and L-cells can lead to a general ‘reset’ of their function to potentially allow for a return to an appropriate endogenous secretion of both GLP-1 and GIP; thus, leading to improved glucose regulation. Finally, ablation of the cells at the duodenum and/or stomach can reduce the anti-incretin release. The lack of the anti-incretin (DPP-4) release will allow for continued signaling for insulin secretion by GLP-1 and GIP.

### D. Other Enteroendocrine Hormones

Although GLP-1 and GIP may be the key hormones in glucose modulation, there are various other enteroendocrine hormones known to contribute to glucose modulation. Enteroendocrine play different roles during different states of digestion, ranging from fasting, during nutrient consumption, and postprandial. One example is Ghrelin which is secreted by A-cells which are found in the stomach corpus.

Ghrelin is one of the circulating peptides, which stimulates appetite and regulates energy balance, and thus is connected to obesity and T2DM. Both basic research and genetic association studies have revealed association between the ghrelin gene and obesity, metabolic syndrome or T2DM. Postprandial plasma ghrelin is suppressed proportional to meal calorie content in normal weight but not in obese subjects, which suggest that food intake fails to suppress ghrelin levels in obese humans. Ghrelin secretion may be decreased after RYGB because of denervation of autonomic input to ghrelin cells in the stomach. Ablation of the mucosa in the stomach may disrupt the Ghrelin secretion in the duodenum and/or signaling of the ghrelin receptors.

FIG. 15 is an image of duodenal tissue which has undergone cryoablation 4 days previous and next to duodenal tissue which has not undergone cryoablation. The image is highly magnified histography where in the treated portion includes the treated mucosa, treated villus and crypts which include partial sloughing and regeneration.

FIG. 15, H&E stain, is an image of duodenum (partial thickness), which demonstrates the edge of the cryoablation site at Day 4. Note the untreated intact mucosal epithelium that this viable (black circle), and the area of cryoablation with sloughing of the much of the mucosal epithelium and necrosis and loss of submucosal glands (dashed black circle). Normal viable submucosal glands are indicated by “SM”, and the lumen of the duodenum by “L”. The area of cyroablation demonstrates loss of the treated area of mucosal epithelium, representing the villi and crypts, which include mucosal epithelial cells, secretory goblet cells, and the EECs.

FIG. 16, H&E stain, is an image of the duodenum (partial thickness), which demonstrates the edge of the cryoablation site at Day 30. Note the untreated intact mucosal epithelium that is viable (black circle), and the area of mucosal regeneration, with early formation of villi and crypts (dashed black circle). This area of mucosal regeneration represents proliferation of mucosal epithelial cells, secretory goblet cells, and expected EECs. The lumen of the duodenum is indicated by “L”.

### Disruption of Enteric Nervous System:

The upper small intestine is highly innervated by the afferent nerves which forms part of neural circuit that is responsible for motility, secretion, inflammatory response, satiety, and insulin regulation. The parasympathetic nervous system makes up the enteric nervous system (ENS), which is responsible for the complex signaling by the enteroendocrine hormones. The vagal afferent innervation of the submucosal plexus consists of fibers that pass through the submucosa to the level of the muscularis mucosa, where the neurites then arborize extensively. The plexus includes chemoreceptors and mechanoreceptors which provide input signals to the ENS. As a whole, these afferent neurons transmit information to determine the gut response, such as motility, intestinal barrier function, and epithelial secretion. The ENS transmits information to the central autonomic nervous system through afferent nerves of the small intestine which correspond with specific areas in the brain (the well-known gut-brain axis). Additionally, this system is a key regulator of insulin production and glycemic management. Dysfunction of the signaling pathway can lead to improper insulin production, leading to T2DM.

Cryotreatment of peripheral nervous tissue has been commonly performed for various medical therapies (e.g., pain management and movement disorders). Different strategies in cryoablation of the nerve cause varying levels conduction disruption, ranging from temporary to permanent denervation. The proposed device can disrupt the nerve terminals and plexus located in the mucosa and/or the submucosa. The cryotreatment can induce a process known as Wallerian degeneration. This process involves the degeneration of the nerve axon and disruption of nerve conduction signals. This disruption can modify enteric nervous response and improve insulin sensitivity like the effects seen in duodenal bypass. Appropriate cryotreatment of the nerve may allow the epineurial structure to remain intact; thereby, providing scaffolding for axonal regeneration to occur. It is believed that the disruption to the nerves in the duodenum can disrupt the signal; like what is observed in RYGB and vertical sleeve gastrectomy patients who show improved energy homeostasis and metabolism by manipulation of vagal afferent fibers of the ENS.

Temperature decrease to the submucosa is therapeutic in the sense that the energy delivery facilitates modulation of the submucosal plexus (partial or reversable ablation, blocking, stimulation) while leaving critical microvascular and structural proteins intact which are necessary for regeneration. Additionally, cryoablation has been observed to not produce coagulative effect; thus, resulting in a lower incidence of thrombus formation as compared to high-temperature based ablation.

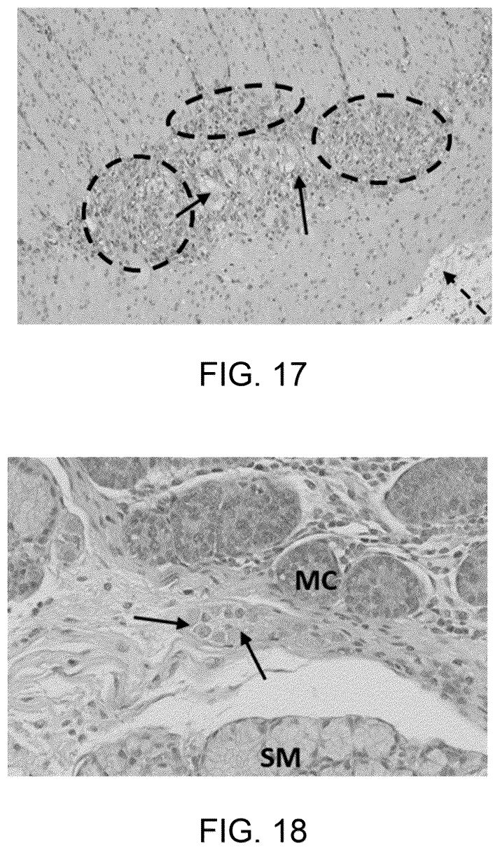

FIG. 17, H&E, is a high power image of a myenteric plexus between the inner and outer smooth muscle layers of the muscularis externa, adjacent to a cryoablation treatment site, at Day 4. Ganglia within the plexus exhibit mild to moderate vacuolation of the cytoplasm (black arrows), which histologically appears as clear cytoplasmic spaces. This vacuolar change represents a non-specific degenerative process, indicative of injury within the enteric nervous system associated with cryoablation. There is a mild to moderate inflammatory infiltrate surrounding the plexus (lymphocytes and lesser plasma cells) indicated by black dashed circles. The serosal surface is indicated by the dashed black arrow. Adjacent smooth muscle of the muscularis externa is normal and viable.

FIG. 18 is a high power image of a submucosal plexus, adjacent to a cryoablation treatment site at Day 4. Ganglia within the plexus exhibit mild vacuolation of the cytoplasm (black arrows), consistent with a degenerative change, indicative of injury within the enteric nervous system associated with cryoablation. Note, normal adjacent submucosal glands (SM), and mucosal crypts. (MC)

### Cryogenic Fluid Delivery Channel Assembly Without the Use of a Central Rail

In another aspect, the present disclosure provides a cryogenic fluid delivery channel assembly that does not require the use of a central rail or any rail.

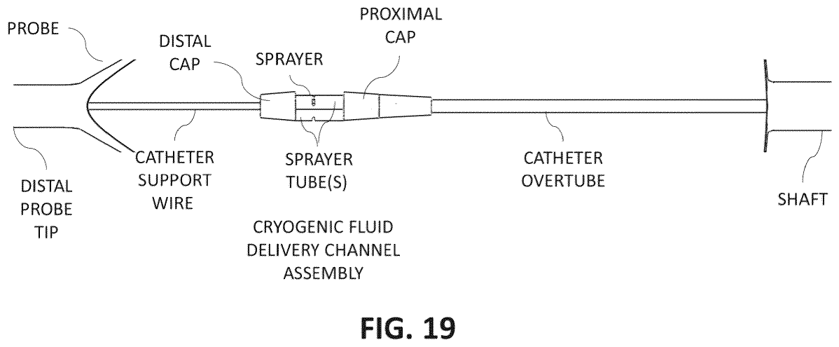

The cryogenic fluid delivery channel assembly can be adapted for use with one or more delivery channels for the distribution of a cryogenic fluid to a target region, target area, treatment zone, or the like. The one or more delivery channels can be arranged, bundled, and/or otherwise organized with the cryogenic fluid delivery channel assembly in various arrangements as disclosed herein. The one or more delivery channels can be combined at their terminal (delivery) ends with a sprayer for delivery of the cryogenic fluid in a desired pattern of distribution. Various arrangements and aspects of the sprayer and delivery channel(s) are described below and in FIGS. 19-52. For example, the sprayer can be part of a cryogenic fluid delivery channel assembly and various types of sprayer tubes or arrangements.

The cryogenic fluid delivery channel assembly can be used in various medical devices and methods such as in cryogenic catheter probes, cryogenic ablation systems, and methods for their use for selectively ablating of the mucosa and submucosa in the gastrointestinal tract, including, inter alia, for the treatment of Type 2 Diabetes, obesity, and other metabolic and medical conditions.

In one aspect, the present disclosure provides a cryogenic catheter including a probe at the distal end of the catheter, a shaft at the proximal end of the catheter, and a cryogenic fluid delivery channel assembly operably coupled to and/or associated with the probe and shaft. The cryogenic fluid delivery channel assembly includes a cryogenic sprayer component, a catheter support wire, and a catheter overtube. The cryogenic sprayer component includes at least one sprayer tube and at least one delivery channel. The at least one sprayer tube includes at least one sprayer through which a cryogenic fluid transported within the at least one delivery channel can be delivered to one or more target areas or treatment zones.

In one embodiment of the cryogenic catheter of the present disclosure, the sprayer tube is securely fastened between a distal cap and a proximal cap distal cap. Examples of these types of cryogenic catheter of the present disclosure are illustrated in FIGS. 19-39.

In one embodiment of the cryogenic catheter of the present disclosure where the sprayer tube is securely fastened between a distal cap and a proximal cap distal cap, the cryogenic sprayer component includes multiple sprayer tubes each containing a single cryogenic fluid delivery channel. Examples of these types of cryogenic catheter of the present disclosure are illustrated in FIGS. 19-35. In certain embodiments of these types of cryogenic catheters, the design incorporates multiple cut sprayer tubes secured between a distal cap and a proximal cap, establishing a rigid configuration that effectively eliminates any flexure of the sprayer openings. Such arrangements contribute to the creation of the cryogenic fluid delivery channel assembly. In certain examples of this type of cryogenic catheter of the present disclosure, each individual delivery channel is carefully threaded through the proximal cap into their respective sprayer where the delivery channel concludes at the sprayer. In certain embodiments of this type of cryogenic catheter of the present disclosure, there are multiple sprayer tubes with one delivery channel per sprayer tube.

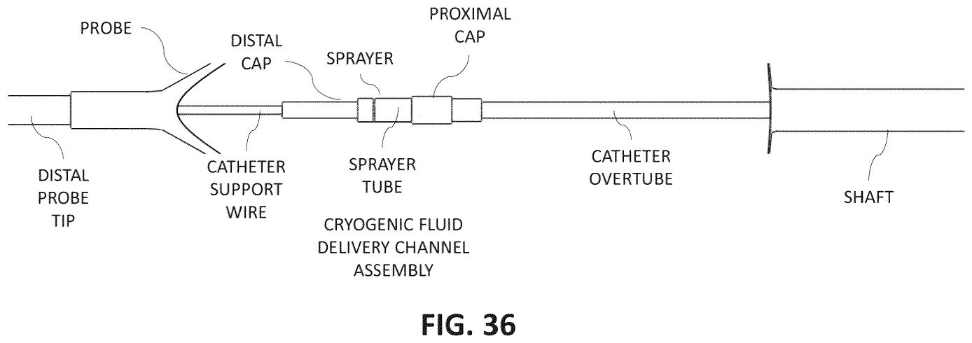

In another embodiment of the cryogenic catheter of the present disclosure where the sprayer tube is securely fastened between a distal cap and a proximal cap distal cap, the cryogenic sprayer component includes a single sprayer tube containing multiple cryogenic fluid delivery channels. Examples of these types of cryogenic catheter of the present disclosure are illustrated in FIGS. 36-39. In certain embodiments of these types of cryogenic catheters, this design seamlessly incorporates a single sprayer tube. Holes can be drilled to accommodate the delivery channels and laser cut openings that constitute individual sprayers. The proximal half of the sprayer tube or a secondary piece of the sprayer tube serves to discreetly guide the delivery channels to the sprayers. The delivery channels are intricately thread through the sprayer tube, ultimately converging at the sprayers within the cryogenic fluid delivery channel assembly. In certain embodiments of this type of cryogenic catheter of the present disclosure, there is one sprayer tube with multiple delivery channels in the one sprayer tube.

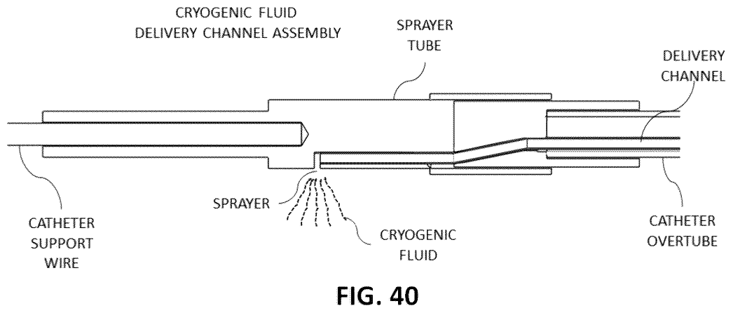

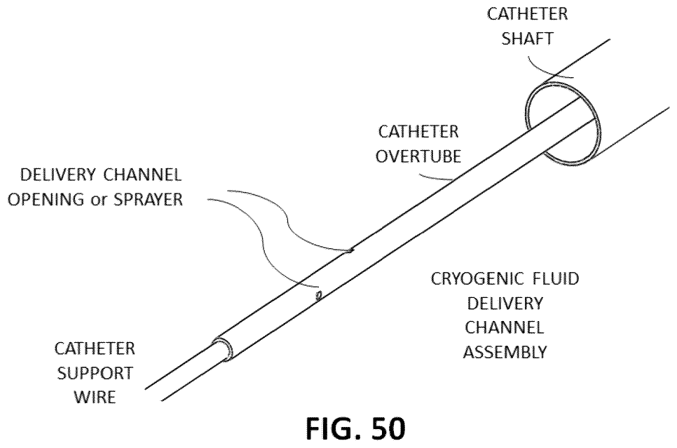

In one embodiment of the cryogenic catheter of the present disclosure, the cryogenic sprayer component includes a single sprayer tube containing one or more cryogenic fluid delivery channels. Examples of these types of cryogenic catheter of the present disclosure are illustrated in FIGS. 40-52. In certain embodiments of these types of cryogenic catheters, this design seamlessly incorporates a single sprayer tube which is made of one material. Holes that are laser cut or extruded can be drilled to accommodate the delivery channels and laser cut openings that constitute individual sprayers. In certain embodiments of this type of cryogenic catheter of the present disclosure, this design adopts a spray tube configuration without internal moving components within the sprayer tube. In this embodiment, no movement is required to select each sprayer location. In this embodiment, the delivery channels are cut and precisely aligned to the sprayer opening. Additionally, in this embodiment, the delivery channel/catheter overtube is constructed from a single multi lumen tubing notched at each sprayer location utilizing adhesive to isolate the spray to a single opening. FIG. 50 is a particular embodiment where there is no sprayer tube, but where there are sprayers notched/laser cut into the catheter overtube so that the delivery channel includes individual sprayers directly into the delivery channel.

In various embodiments, the at least one sprayer includes multiple sprayers radially aligned, radially staggered, or both radially aligned and staggered in reference to their positions on the sprayer tube.

In various embodiments, the cryogenic fluid delivery channel assembly is configured for translational movement longitudinally between the distal and proximal ends of the catheter.

In various embodiments, the translational movement occurs without requiring a central rail component.

In various embodiments, a distal probe tip is attached to the distal end of the probe.

In various embodiments, the cryogenic fluid delivery channel assembly comprises 1 to 20 delivery channels.

In various embodiments, the cryogenic fluid delivery channel assembly comprises a number of delivery channels selected from the group consisting of 1, 2, 3, 4, 5, 6, 7, and 8 delivery channels.

In various embodiments, the sprayers are configured to release the cryogenic fluid either in unison or independently of one another.

In various embodiments, each sprayer is configured to release the cryogenic fluid in a different treatment zone, including in different treatment zones along the probe.

In various embodiments, the catheter is configured for selective ablation in the mucosa and submucosa of a gastrointestinal tract of a subject.

In various embodiments, the delivery channels are independently controlled and turned to an on or off position.

In various embodiments, the treatment tissue, treatment zone, or treatment area comprises mucosal and/or submucosal tissue of the large intestine, small intestine, stomach, esophagus, rectum, and anus.

In another aspect, the present disclosure provides a cryogenic ablation system. The cryogenic ablation system includes a cryogenic catheter as disclosed herein; and a controller configured to control the functionality of the cryogenic catheter.

In various embodiments, the catheter shaft is connected to a handle.

In various embodiments, the system further comprises a pressure detection tube disposed within the cryogenic catheter probe or catheter shaft.

In various embodiments, the at least one sprayer is connected to the outside of the distal end of the at least one delivery channel.

In various embodiments, each delivery channel is fluidly connected to a delivery channel control valve and a reservoir of cryogenic fluid whereby the delivery channel can be controlled by actuation of each delivery channel control valve. As used herein, the term “control valve” may also be referred to as a “flow valve” and the like.

In various embodiments, each delivery channel is associated with a sprayer which allows for partial restriction of the cryogenic fluid and a delivery channel control valve controls release of the cryogenic fluid from a cryogenic supply block into each delivery channel.

In various embodiments, the cryogenic catheter probe is placed into an expanded state upon release of cryogenic fluid into the inside of the cryogenic catheter probe.

In various embodiments, the controller independently controls deliver of cryogenic fluid to each delivery channel via control valves at the proximal end of each delivery channel, and/or wherein a reservoir system allows for large ablation areas up to 10 cm and beyond of tissue in a partial-circumferential or full-circumferential ablations.

In various embodiments, the controller comprises one or more variable controller parameters used to control the functional assembly.

In various embodiments, the system further comprises at least one sensor constructed and arranged to produce a sensor signal.

In various embodiments, the controller is configured to perform closed-loop energy delivery to the functional assembly based on the sensor signal.

In another aspect, the present disclosure provides a method of performing cryogenic ablation of mucosal tissue and/or of both mucosal tissue and submucosal tissue in the gastrointestinal tract of a subject. This method includes the steps of: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In various embodiments, the target treatment region comprises mucosal tissue and/or both mucosal and submucosal tissue of the large intestine, small intestine, stomach, esophagus, rectum, or anus of the subject.

In various embodiments, treating the target treatment region comprises

performing a series of tissue ablation steps, each comprising ablation of an axial length of the large intestine, small intestine, stomach, esophagus, rectum, or anus of the subject, wherein each ablation step is optionally preceded by a tissue expansion step.

In various embodiments, the method further comprises adjusting at least one variable controller parameter based on the sensor signal.

In another aspect, the present disclosure provides a use of a cryogenic ablation system in a method of performing cryogenic ablation of mucosal tissue and/or of both mucosal tissue and submucosal tissue in the gastrointestinal tract of a subject, where the method comprises: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In another aspect, the present disclosure provides a cryogenic ablation system for use in a method of performing cryogenic ablation of mucosal tissue and/or of both mucosal tissue and submucosal tissue in the gastrointestinal tract of a subject, where the method comprises: (a) providing a cryogenic ablation system as disclosed herein; (b) contacting the cryogenic catheter probe of the system with a target treatment region of the gastrointestinal tract of the subject; and (c) releasing a cryogenic fluid from the at least one sprayer to treat the target treatment region by cryogenically ablating at least a portion the mucosal tissue or ablating at least a portion of both the mucosal and submucosal tissue of the target treatment region.

In another aspect, the present disclosure provides a method for performing a medical procedure in a small intestine and/or stomach of a patient in need of the medical procedure. This method includes the steps of: (a) providing a cryogenic ablation system as disclosed herein; (b) contacting the cryogenic catheter probe of the system with a target treatment region of the small intestine and/or stomach of the patient; and (c) releasing a cryogenic fluid from the at least one sprayer to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In various embodiments, treating the target treatment region comprises performing a series of tissue ablation steps, each comprising ablation of an axial length of the small intestine or stomach tissue, where each ablation step is optionally preceded by a tissue expansion step.

In various embodiments, the method further comprises adjusting at least one variable controller parameter based on the sensor signal.

In certain embodiments, the cryogenic fluid delivery channel assembly encompasses one or more delivery channels, one or more sprayers, one or more sprayer tubes or no sprayer tubes, where each delivery channels runs into a sprayer tube and a sprayer tube may have one or more delivery channels or the delivery channels directly delivery cryogenic fluid to the probe inner surface. In certain embodiments, each sprayer tube will have one or more sprayers. In certain embodiments, each delivery channel may have one or more delivery channel openings.

Various embodiments and arrangements of the cryogenic catheter, cryogenic catheter probe, cryogenic fluid delivery channel assembly, and cryogenic ablation system of the present disclosure are illustrated in FIGS. 19-52 and further described herein below.

FIG. 19 is an embodiment of the catheter configured sectioned to visualize the probe's components including the probe, the distal probe tip, the catheter shaft, and the cryogenic fluid delivery channel assembly which includes the distal cap, the proximal cap, the sprayer, sprayer tube(s), the catheter support wire, and the catheter overtube. FIG. 20 is an embodiment of the catheter configured sectioned to visualize the probe's components in a perspective view including the probe, the distal probe tip, the catheter shaft, and the cryogenic fluid delivery channel assembly with the catheter support wire and the catheter overtube highlighted. The cryogenic fluid delivery channel assembly which encompasses one or more delivery channels, one or more sprayers, one or more sprayer tubes, a proximal cap, and a distal cap where each delivery channels runs into a sprayer tube and a sprayer tube may have one or more delivery channels. The sprayer tube(s) is equal to or has a slightly smaller or larger diameter than a delivery channel to eliminate or minimize the phase change of the liquid. Each sprayer tube will have one or more sprayers. Each sprayer may be laser cut or created by other methods. Attached to the distal cap is a catheter support wire which is extended most distal within the probe. The catheter support wire extends into the distal probe tip. Attached to the proximal cap is a catheter overtube which extends inside the catheter shaft for the full length of the catheter shaft.

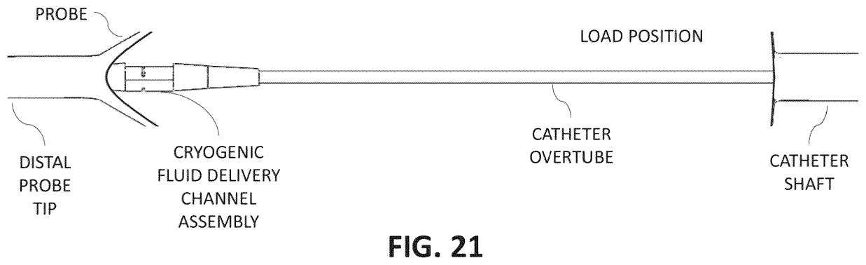

FIG. 21 is an embodiment of the catheter configured in the load position focused and sectioned to visualize the probe's components including the probe, the distal probe tip, the catheter shaft, and the cryogenic fluid delivery channel assembly with the catheter overtube highlighted. The catheter support wire is extended most distal within the distal probe tip and is not visualized, which maximizes the probe's longitudinal characteristic elongating the probe to facilitate an easy introduction of the catheter into and through the endoscope. In this embodiment, the cryogenic fluid delivery channel assembly is also extended to the most distal within the probe.

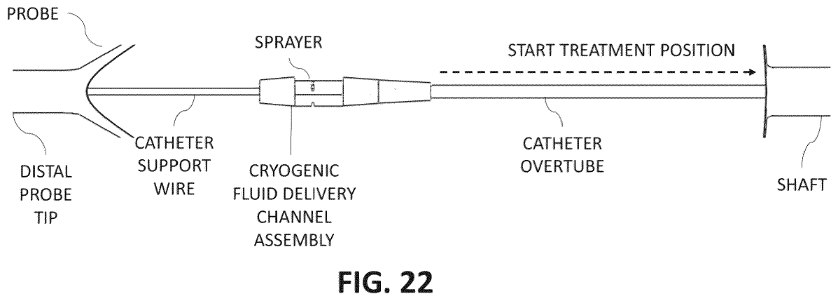

FIG. 22 is an embodiment of the catheter configured in the start treatment position focused and sectioned to visualize the probe's components including the probe, the distal probe tip, the catheter shaft, and the cryogenic fluid delivery channel assembly with a sprayer, the catheter support wire, and the catheter overtube. The catheter has been inserted into the endoscope, fluidly coupled to the controller, and the cryogenic fluid delivery channel assembly has moved to the start treatment position.

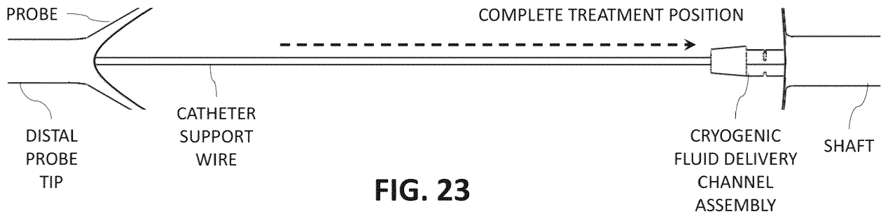

FIG. 2 is an embodiment of the catheter shown in the complete treatment position that has been focused, sectioned to visualize the probe's components including the probe, the distal probe tip, the catheter support wire, the catheter overtube, the catheter shaft, and the cryogenic fluid delivery channel assembly with a sprayer, the catheter support wire and the catheter overtube highlighted. FIG. 23 illustrates the cryogenic fluid delivery channel assembly traversing from the start treatment position to the complete treatment position. The catheter support wire provides longitudinal support while the cryogenic fluid delivery channel assembly is traversing within the probe. The cryogenic fluid delivery channel assembly movement is controlled by a motor, which may be a geared motor with an optical sensor or magnetic reader that translates the cryogenic fluid delivery channel assembly at a precise rate, direction, and location. Once the treatment cycle is complete, the cryogenic fluid delivery channel assembly may be repositioned within the probe. A treatment cycle may be in the distal to proximal direction or proximal to distal direction. During the treatment cycle, the cryogenic fluid delivery channel assembly is held rotationally fixed, ensuring a repeatable application of cryogenic fluid to the desired tissue.

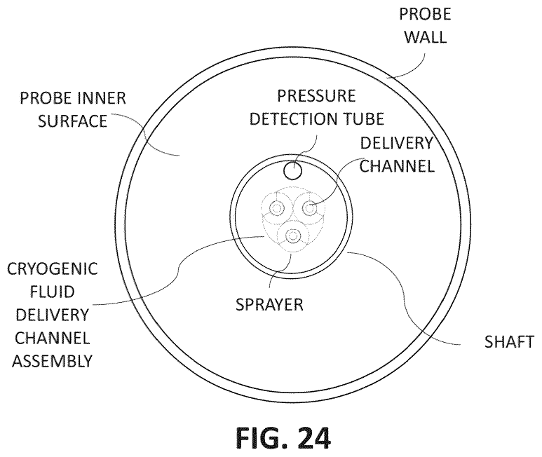

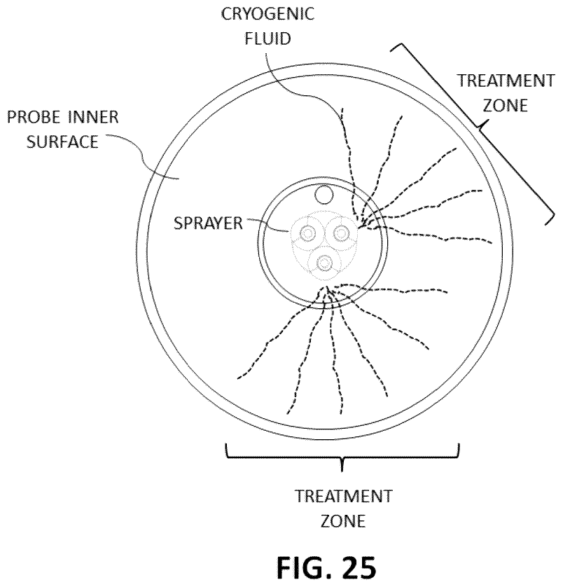

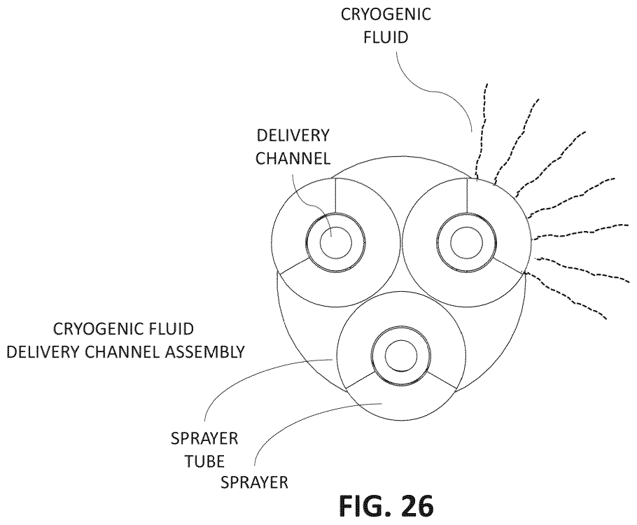

FIG. 24 is a cross-sectional view of the embodiment of the catheter at the distal portion of the probe including the probe wall, the probe inner surface, the catheter shaft, the pressure detection tube, and cryogenic fluid delivery channel assembly with a delivery channel and a sprayer highlighted. This catheter has been configured with 3 delivery channels and 3 sprayers. The cryogenic fluid delivery channel assembly may have one or more delivery channels and one or more sprayers. FIG. 25 is an embodiment where the cryogen fluid is directed from sprayer(s) outward into the probe inner surface to the probe wall which is in intimate contact with the tissue of the gastrointestinal tract. In this embodiment, each sprayer may be configured to apply a thin blade of cryogenic fluid to one third (⅓) of the probe wall inner circumference, greater than one third (⅓) of the probe wall inner circumference, or less than one third (⅓) of the probe wall inner circumference. This blade can be traversed across the probe at any speed within the motor's capability. In this embodiment, two of the three sprayers are delivering cryogenic fluid to the probe inner surface, and each sprayer delivers cryogenic fluid to approximately one third (⅓) of the probe wall inner circumference. Two treatment zones are illustrated. This blade can be traversed across the probe at any speed. Referring to FIG. 25, in this embodiment, two of the three sprayers are applying a blade of cryogen fluid into the probe inner surface and to the inner portion of the probe wall. The invention can be configured so one or more sprayers can be configured to simultaneously apply cryogen fluid based on the algorithm selected and controlled by the control system. FIG. 26 is a cross-section view of the cryogen fluid delivery channel assembly including the delivery channel(s), sprayer tube(s), and a sprayer(s). In this embodiment, the application of cryogen fluid from one sprayer is illustrated.

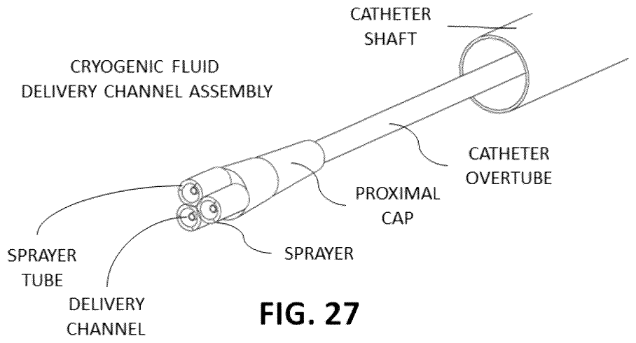

FIG. 27 is a perspective view of the catheter shaft and the cryogenic fluid delivery channel assembly. The cryogenic fluid delivery channel assembly may encompass one or more delivery channels, one or more sprayers, and one or more sprayer tubes. The catheter overtube is connected to the proximal cap and the catheter support wire is connected to the distal cap. The cryogenic fluid delivery channel assembly encompasses the delivery channels and the sprayer tubes, and this section view of the cryogenic fluid delivery channel assembly is at the location of the sprayers. The delivery channels run inside the catheter overtube and at the cryogenic fluid delivery channel assembly, one or more delivery channels may be located in a sprayer tube.

FIG. 28 is a perspective view and close up of the cryogenic fluid delivery channel assembly. The cryogenic fluid delivery channel assembly may encompass one or more delivery channels, and one or more sprayers. The exploded view of the catheter overtube shows the multiple delivery channels that transition into the sprayer tube(s) and in fluid communication with the sprayers. The catheter support wire is also illustrated.

FIG. 29 is a cross-sectional view of the cryogenic fluid delivery channel assembly. In this view, one cryogenic delivery channel is illustrated within the catheter overtube where one or more delivery channels may be present and each delivery channel runs independent through the proximal cap into a sprayer tube and to a sprayer. One or more delivery channels may run in one sprayer tube. Each delivery channel passes through the inside of the proximal cap and may transition at a slight angle orienting the delivery channel toward the outer portion of the cryogenic fluid delivery channel assembly. Each delivery channel continues into a sprayer tube where one or more delivery channels continue into a sprayer tube. The sprayer tube(s) is equal to or has a slightly smaller or larger diameter than a delivery channel to eliminate or minimize the phase change of the liquid. The delivery channel ends in the sprayer tube where the delivery channel may end proximal to the location of the sprayer, distal to the location of the sprayer, or at the location of the sprayer. The sprayer redirects the cryogenic fluid which runs in an axial direction down the delivery channel. At the sprayer, the cryogenic fluid is redirected to flow perpendicular to the axis of the delivery channel. The sprayer configuration will determine the cryogenic fluid pattern which can be customized to spray into the probe inner surface based on the sprayer configuration.

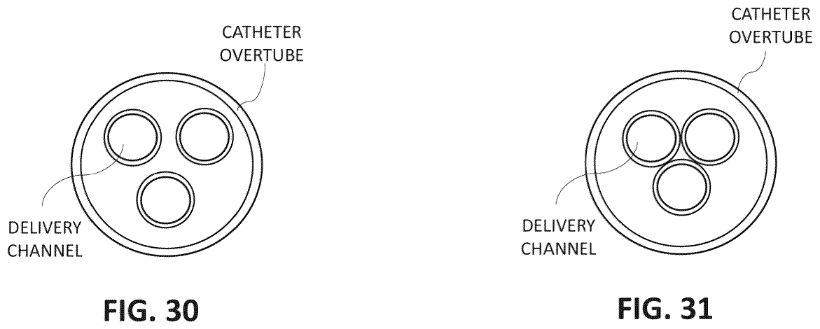

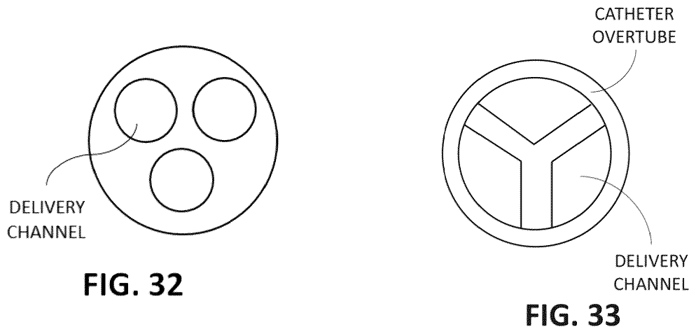

FIG. 30 is a cross-sectional view of the catheter overtube. One or more delivery channels are within the catheter overtube, and each delivery tube runs independent of the other delivery tubes. FIG. 31 is a cross-sectional view of the catheter overtube. One or more delivery channels are within the catheter overtube, and each delivery channel runs independent of the other delivery channel into the sprayer tube and through the sprayer. Each delivery channel is positioned independently or bound together. The binding may be shrink-tubing or other tubing/material that conforms to the shape of the combined delivery channels as a whole or at various positions along the length of the catheter shaft or the binding of shrink tubing or other tubing that conforms to the shape of the combined delivery channels along the complete length of the delivery channels which are inside the catheter overtube. As shown in FIG. 32, in certain embodiments, one or more delivery channels are encased within an otherwise solid catheter overtube, or one or more delivery channels are extruded from within an otherwise solid catheter overtube making a one-piece catheter overtube with one or more delivery channels. Referring to FIG. 33, the catheter overtube may be extruded with one or more non-circular shaped delivery channels.

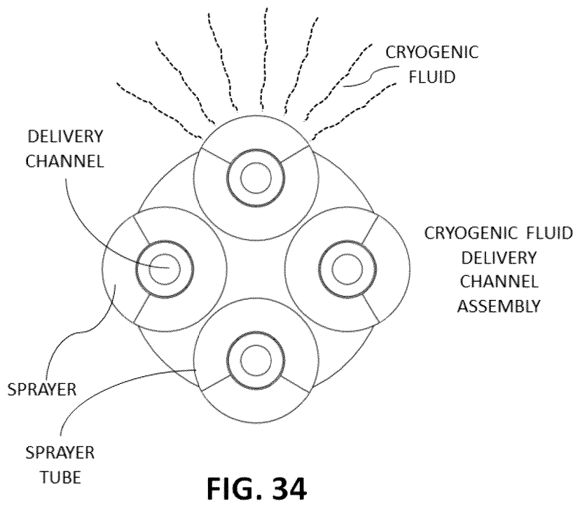

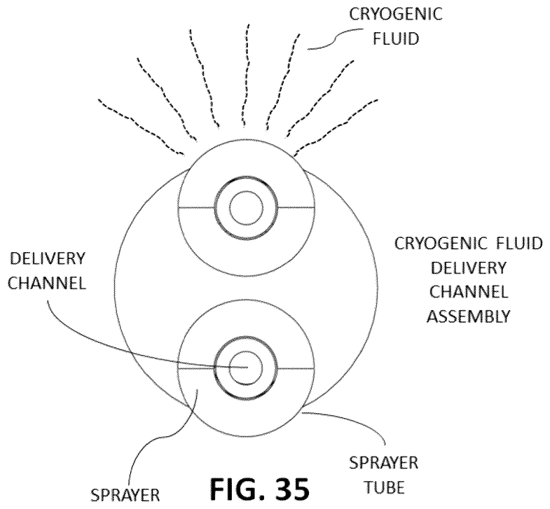

FIG. 34 is a cross-sectional view of the cryogenic fluid delivery channel assembly. In this embodiment, the cryogenic fluid delivery channel assembly is configured with 4 delivery channels, 4 sprayer tubes, and 4 sprayers are present. Each sprayer tube may have one or more delivery channels and one or more sprayers. The delivery channel may be located anywhere within the sprayer tube. Each sprayer may be configured to apply a blade of cryogen fluid to one fourth (¼) of the probe wall inner circumference which is in intimate contact with the tissue of the gastrointestinal tract. In this embodiment, each sprayer may be configured to apply greater than one-fourth (¼) of the probe wall inner circumference or less than one-fourth (¼) of the probe wall inner circumference. The cryogenic fluid delivery channel assembly of the present disclosure can be configured so one or more sprayers can be configured to simultaneously apply cryogen fluid based on the algorithm selected and controlled by the control system.

FIG. 35 is a cross-sectional view of the cryogenic fluid delivery channel assembly. In this embodiment, the cryogenic fluid delivery channel assembly is configured with 2 delivery channels, 2 sprayer tubes, and 2 sprayers. The cryogenic fluid delivery channel assembly may have one or more delivery channels and one or more sprayers. The delivery channel may be located anywhere within the sprayer. Each sprayer may be configured to apply a blade of cryogen fluid to one half (½) of the probe wall inner circumference which is in intimate contact with the tissue of the gastrointestinal tract. In this embodiment, each sprayer may be configured to apply greater than one-half (½) of the probe wall inner circumference or less than one-half (½) of the probe wall inner circumference. The invention can be configured so one or more sprayers can be configured to simultaneously apply cryogen fluid based on the algorithm selected and controlled by the control system.

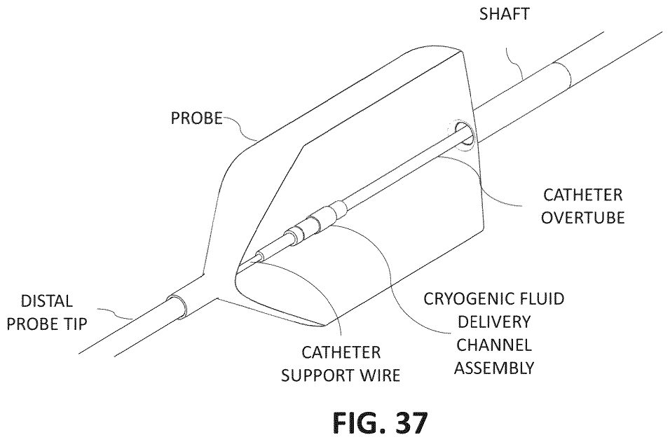

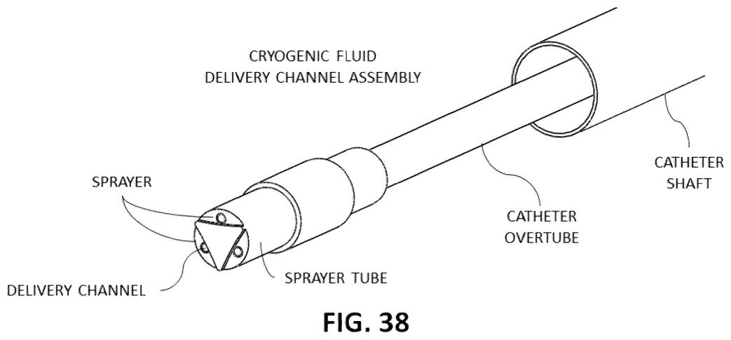

Referring to FIG. 36, FIG. 37, and FIG. 38, in certain embodiments, the catheter is configured sectioned to visualize the probe's components which include the probe, distal probe tip, catheter shaft, and the cryogenic fluid delivery channel assembly, which encompasses one or more delivery channels, one or more sprayers, one or more sprayer tubes, a proximal cap, a distal cap, a catheter support wire, and a catheter overtube where each delivery channels runs into a sprayer tube and a sprayer tube may have one or more delivery channels. The sprayer tube(s) is equal to or has a slightly smaller or larger diameter than a delivery channel to eliminate or minimize the phase change of the liquid. The delivery channel may be located anywhere within the sprayer. Each sprayer tube will have one or more sprayers. Each sprayer may be laser cut. The catheter support wire is attached to the distal cap and extends most distal within the probe. The catheter support wire extends into the distal probe tip and is attached to the cryogenic fluid delivery channel assembly at the most distal portion of the cryogenic fluid delivery channel assembly. The catheter overtube is attached to the proximal cap.

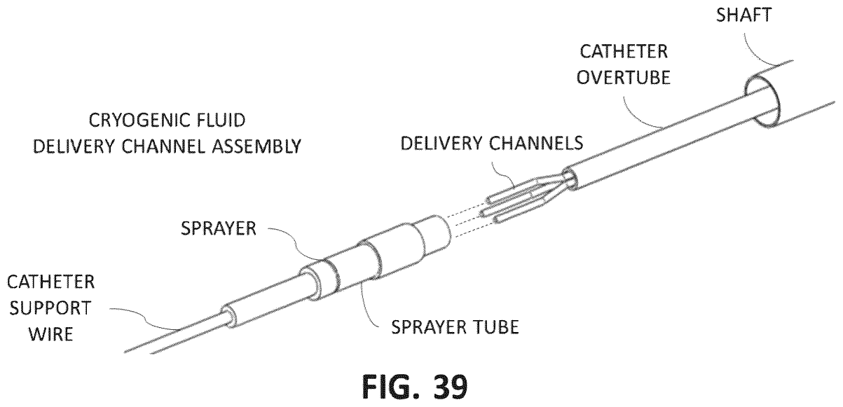

FIG. 39 is a perspective view which the cryogenic fluid delivery channel assembly. The cryogenic fluid delivery may encompass one or more delivery channels, one or more sprayers, and one sprayer tube where there may be one or more sprayer tubes. The exploded view of the catheter overtube shows the multiple delivery channels that transition into the sprayer tube, through one sprayer tube and in fluid communication with the sprayers.

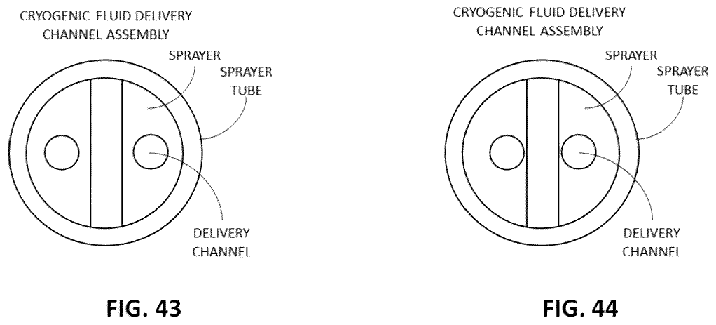

FIG. 40 is a cross-sectional view of the cryogenic fluid delivery channel assembly. In this view, one cryogenic delivery channel assembly is illustrated within the catheter overtube where one or more delivery channels may be present and each delivery channel runs independently through the sprayer tube into one sprayer tube and to each sprayer. Each delivery channel passes through the inside of the sprayer tube and may transition at a slight angle orienting the delivery channel toward the outer portion of the cryogenic delivery channel assembly. Referring to FIG. 43 and FIG. 44, the transition and angle of the delivery channel may be such that the end of the delivery channel ends at different locations within the sprayer. Referring to FIG. 40, each delivery channel continues into the sprayer tube where sprayers are laser cut or by some other process. One or more delivery channels end at the location of each sprayer. The sprayer redirects the cryogenic fluid which runs in an axial direction down the delivery channel. At the sprayer, the cryogenic fluid is redirected to flow perpendicular to the axis of the delivery channel. The sprayer configuration will determine the cryogenic fluid pattern which can be customized to spray into the probe inner surface based on the sprayer configuration. The delivery channel may be located anywhere within the sprayer.

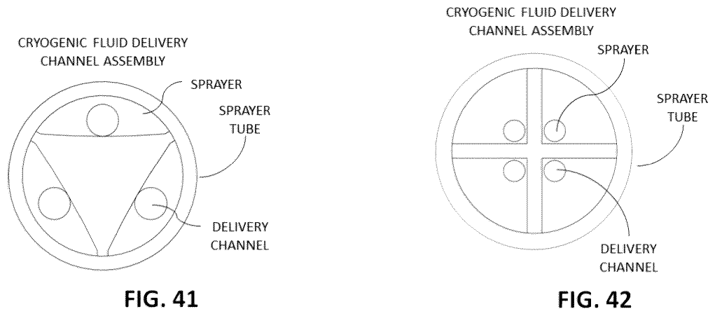

FIG. 41 is a cross-sectional view of the cryogenic fluid delivery channel assembly where 3 delivery channels, 3 sprayers, and one sprayer tube are present. The delivery channel may be located anywhere within the sprayer. The cryogenic fluid delivery channel assembly may have one or more delivery channels and one or more sprayers. In this embodiment, each sprayer may be configured to apply a blade of cryogen fluid to approximately one third (⅓) of the probe wall inner circumference which is in intimate contact with the tissue of the gastrointestinal tract. Each sprayer may be configured to apply more than one-third (⅓) of the probe wall inner circumference or less than one-third (⅓) of the probe wall inner circumference. The cryogenic fluid delivery channel assembly of the present disclosure can be configured so one or more sprayers can be configured to simultaneously apply cryogen fluid based on the algorithm selected and controlled by the control system.

FIG. 42 is a cross-sectional view of the cryogenic fluid delivery channel assembly where 4 delivery channels, 4 sprayers, and 1 sprayer tube are present. The delivery channel may be located anywhere within the sprayer. The cryogenic fluid delivery channel assembly may have one or more delivery channels and one or more sprayers. Each sprayer may be configured to apply a blade of cryogenic fluid to approximately one fourth (¼) of the probe wall inner circumference which is in intimate contact with the tissue of the gastrointestinal tract. Each sprayer may be configured to apply greater than one-fourth (¼) of the probe wall inner circumference or less than one-fourth (¼) of the probe wall inner circumference. The cryogenic fluid delivery channel assembly of the present disclosure can be configured so one or more sprayers can be configured to simultaneously apply cryogen fluid based on the algorithm selected and controlled by the control system.

FIG. 43 is a cross-sectional view of the cryogenic fluid delivery channel assembly where 2 delivery channels, 2 sprayers, and one sprayer tube are present. The delivery channel may be located anywhere within the sprayer. The cryogenic fluid delivery channel assembly may have one or more delivery channels and one or more sprayers. Each sprayer may be configured to apply a blade of cryogen fluid to approximately one half (½) of the probe wall inner circumference which is in intimate contact with the tissue of the gastrointestinal tract. Each sprayer may be configured to apply greater than one-half (½) of the probe wall inner circumference or less than one-half (½) of the probe wall inner circumference. The cryogenic fluid delivery channel assembly of the present disclosure can be configured so one or more sprayers can be configured to simultaneously apply cryogen fluid based on the algorithm selected and controlled by the control system.

FIG. 44 is cryogenic fluid delivery channel assembly where 2 delivery channels, 2 sprayers are present, and 1 sprayer tube. The delivery channel may be located anywhere within the sprayer and this embodiment demonstrates a different location from FIG. 44.

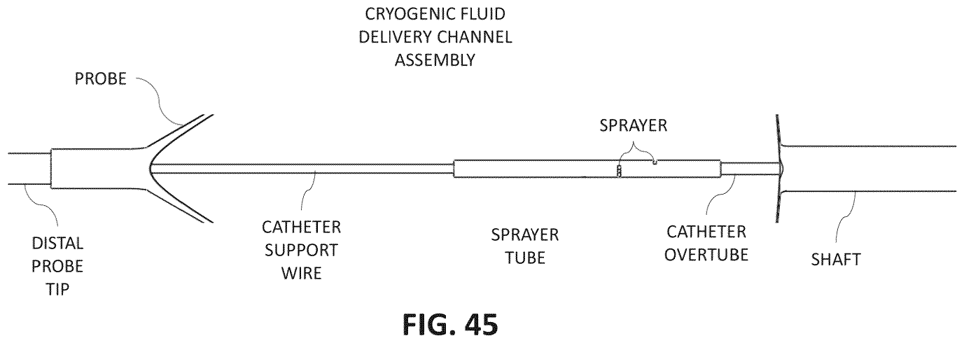

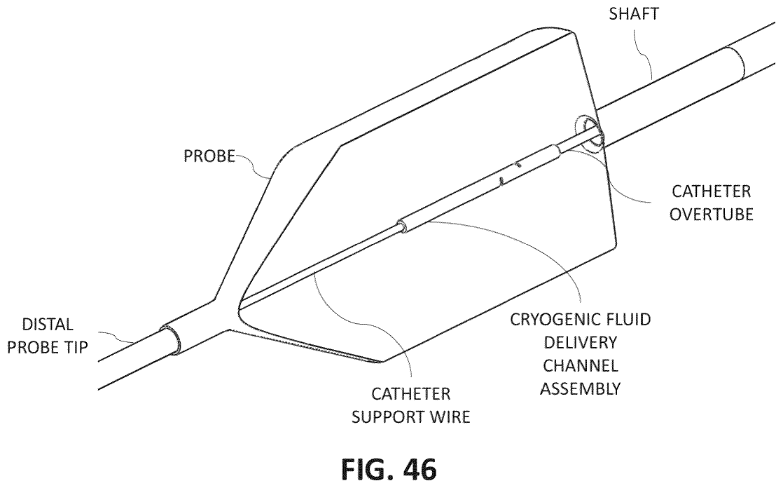

Referring to FIG. 45 and FIG. 46, in certain embodiments, the cryogenic fluid delivery channel assembly includes one or more delivery channels, one or more sprayers, and one or more sprayer tubes. A sprayer tube may have one or more delivery channels. The sprayer tube(s) is equal to or has a slightly smaller or larger diameter than a delivery channel to eliminate or minimize the phase change of the liquid. Each sprayer tube will have one or more sprayers. Attached on the distal end or notch along the length either in the same plane or spaced longitudinally of the sprayer tube is the catheter support wire which is extended most distal within the probe. The catheter support wire extends into the distal probe tip and is attached rigidly or with a flexible member at the most distal portion of the sprayer tube. Attached to the most proximal end of the sprayer tube is a catheter overtube which extends inside the catheter shaft for the full length of the catheter shaft.

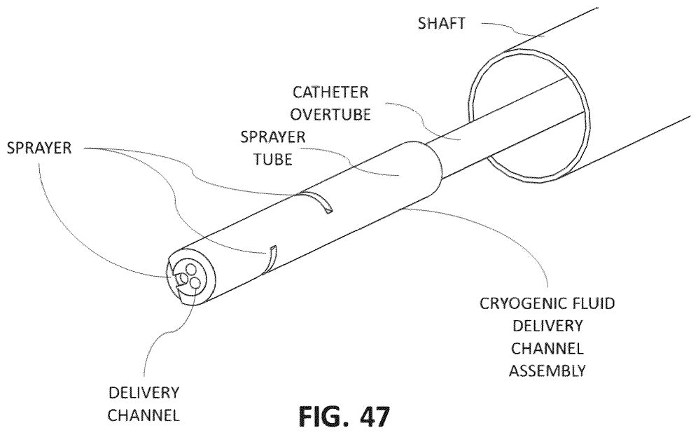

FIG. 47 is a perspective view of the cryogenic fluid delivery channel assembly. The cryogenic fluid delivery channel assembly may encompass one or more delivery channels, one or more sprayers, and one sprayer tube. The section view of the cryogenic fluid delivery channel assembly at the location of the sprayers. The delivery channels run inside the catheter overtube and at the cryogenic fluid delivery channel assembly, one or more delivery channels may be located in a sprayer tube. The sprayer tube will have one or more sprayers. Each sprayer may be laser cut or created by various methods. Attached to the distal end or notch along the length either in the same plane or spaced longitudinally of the sprayer tube is a catheter support wire which is extended most distal within the probe. The catheter support wire extends into the distal probe tip and is attached to the sprayer tube rigidly or with a flexible member at the most distal portion of the cryogenic fluid delivery channel assembly attached to the most proximal end of the sprayer tube is the catheter overtube which extends inside the catheter shaft for the full length of the catheter shaft.

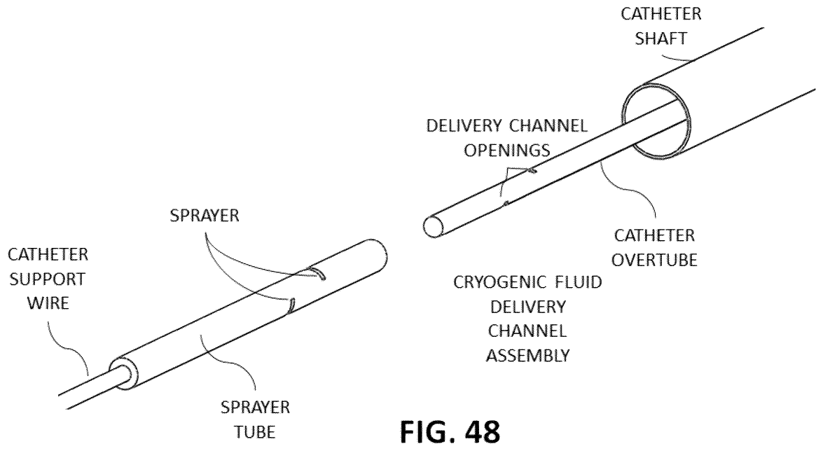

FIG. 48 is a perspective view and close up cryogenic fluid delivery channel assembly. The cryogenic fluid delivery channel assembly may encompass one or more delivery channels, one or more sprayers, and one sprayer tube. One or more delivery channels are within the catheter overtube. The delivery channels are joined or bound together by shrink tubing or other tubing/material that encases one or more delivery channel(s) forming a customizable shape of the combined delivery channels as a whole or the delivery channels are bound together at various positions along the length of the catheter overtube. The exploded view of the catheter overtube shows delivery channel openings along the axis of the catheter overtube and each delivery channel opening aligns with the corresponding sprayer allowing for fluid communication.

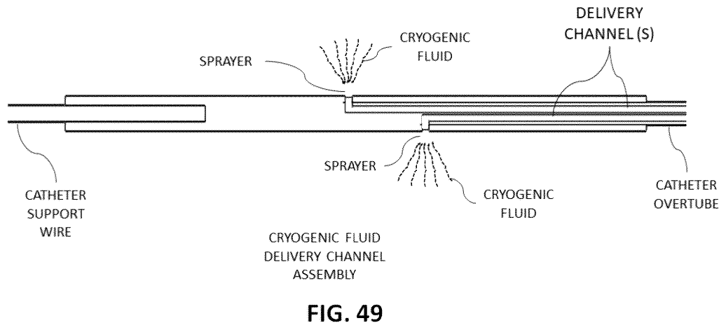

FIG. 49 is a cross-sectional view of the cryogenic fluid delivery channel assembly. In this view, one or more delivery channels are within the catheter overtube where the delivery channels are joined or bound together. Each delivery channel contains one or more delivery channel openings, and each delivery channel opening aligns with the corresponding sprayer in the one sprayer tube creating fluid communication between the delivery channels and the sprayer. The sprayer redirects the cryogenic fluid which runs in an axial direction down the delivery channel. At the sprayer, the cryogenic fluid is redirected to flow perpendicular to the axis of the delivery channel. The sprayer configuration will determine the cryogenic fluid pattern which can be customized to spray into the probe inner surface based on the sprayer configuration.

FIG. 50 is a cross-sectional view of the cryogenic fluid delivery channel assembly. In this embodiment, there is no sprayer tube as shown in FIG. 49. One or more delivery channels are within the catheter overtube where the delivery channels are joined or bound together. Each delivery channel contains one or more delivery channel openings or sprayers and the delivery channel openings or sprayers function as a sprayer and the delivery channel opening(s) form a customized pattern. The delivery channel opening or sprayer redirects the cryogenic fluid which runs in an axial direction down the delivery channel. At the delivery channel opening or sprayer, the cryogenic fluid is redirected to flow perpendicular to the axis of the delivery channel. The delivery channel opening or sprayer configuration will determine the cryogenic fluid pattern which can be customized to spray into the probe inner surface based on the sprayer configuration. Each delivery channel opening or sprayer may be configured to apply a customized pattern of cryogen fluid to the probe wall which is in intimate contact with the tissue of the gastrointestinal tract. The invention can be configured so one or more sprayers or one or more delivery channel openings can be configured to simultaneously apply cryogen fluid based on the algorithm selected and controlled by the control system.

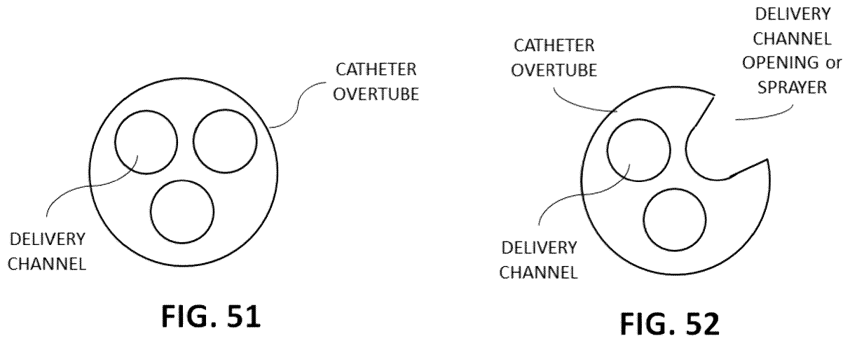

FIG. 51 is a cross-sectional view of the catheter overtube and one or more delivery channels as a single piece. One or more delivery channels are within the catheter overtube, and each delivery channel runs independent of the other delivery channels. The binding may be shrink tubing or other tubing/material that encases one or more delivery channel(s) forming a customizable shape of the combined delivery channels as a whole or at various positions along the length of the overtube.

FIG. 52 is a cross-sectional view of the catheter overtube and delivery channels encased together forming t a single piece with a hole/notch forming a sprayer or delivery channel opening. A customizable hole/notch will determine the cryogenic fluid pattern that will be sprayed to the probe inner surface.

Table B describes various materials and dimensional ranges of certain components, elements, aspects, or items of the devices and systems of the present disclosure. The abbreviation “Ref. No.” refers to any reference numerals included or added to the Figures and/or the present disclosure corresponding to the listed component, element, aspect, or item.

TABLE B

| | Element, Aspect, or | | Ranges | Materials |
|---|---|---|---|---|
| Ref. No. | Item | FIGS. | (Dimensions) | (Source of Materials) |
| | Controller | 1, 3, 4, 6, 7 | NA | NA |
| | Controller Connector | 1, 6, 7 | Custom Machined | PC, Ultem, PEEK, PE, |
| | | | or Molded | PU, Delrin |
| | | | Component | |
| | Load Cap | 3, 4, 6, 7 | ID.1″-2.0″ | Aluminum, SS, Steel, |
| | | | OD.25″ 3.0″ | Nylon, Delrin, PC, PE, |
| | | | Custom Machined | PU, PP |
| | | | or Molded | |
| | | | Component | |
| | Display | 3, 6 | NA | Off the shelf component |
| | Front Panel | 3, 6 | NA | Nylon, Pebax, PC, PE, |
| | | | | PU, PP |
| | Block Body | 4, 6 | NA | Nylon, Pebax, PC, PE, |
| | | | | PU, PP |
| | Mounting Block | 4, 6 | Custom Machined | Nylon, Pebax, PC, PE, |
| | | | or Molded | PU, PP, Aluminum, SS, |
| | | | Component | Steel, Nylon, Delrin, |
| | | | | Rubber |
| | Mounting Wheel | 4, 6 | Custom Machined | Nylon, Pebax, PC, PE, |
| | | | or Molded | PU, Ultem, Aluminum, |
| | | | Component | SS |
| | Motherboard (backside | 6 | NA | PCBA |
| | of display) | | | |
| | Cryogenic Fluid | 6, 7 | Custom Machined | NA |
| | Delivery System | | or Molded | |
| | | | Component | |
| | Cryogenic Supply | 6, 7 | Custom Machined | NA |
| | System | | or Molded | |
| | | | Component | |
| | Cartridge Container | 7 | 8, 12, 14, 16, 24, | Stainless Steels, Steel, |
| | | | 36, 48 grams | Rubber |
| | Catheter Locking | 7 | NA | Off the shelf component |
| | Motor | | | |
| | Cryogenic Supply | 7 | Custom Machined | Aluminum, SS, Steel, |
| | Block | | or Molded | Nylon, Delrin, PC, PE, |
| | | | Component | PU, PP |
| | Delivery Channel Flow | 7 | NA | Off the shelf component |
| | Valve | | | |
| | Exhaust Chamber | 7 | L - 1.0-4.0″ | Aluminum, SS, Steel, |
| | | | H - 1.0″-6.0″ | Nylon, Delrin, PC, PE, |
| | | | W - 1.0″-6.0″ | PU, PP, Foam, open cell |
| | | | Custom Machined | |
| | | | or Molded | |
| | | | Component | |
| | Exhaust Valve | 7 | NA | Off the shelf component |
| | Flow/Pressure PCBA | 7 | NA | Off the shelf, custom |
| | | | | component |
| | High Pressure | 7 | Custom Machined | PC, Ultem, PEEK, PE, |
| | Connector | | or Molded | PU, Delrin |
| | | | Component | |
| | Main Flow Valve | 7 | NA | Off the shelf component |
| | Treatment Motor | 7 | NA | Off the shelf component |
| | Cryogenic Fluid | 19, 20, 21, 22, | ID - .002″-.080″ | Polyimide, PEEK, |
| | Delivery Channel | 23, 24, 26, 27, | OD - .006″-.1″ | Nylon, Pebax, ss, steel, |
| | Assembly | 28, 29, 34, 35, | L - 4″-125″ | nitinol |
| | | 36, 37, 38, 39, | Custom extruded | |
| | | 40, 41, 42, 43, | Component | |
| | | 44, 45, 46, 47, | | |
| | | 49 | | |
| | Catheter Shaft | 19, 20, 21, 22, | Custom Extrusion - | Nylon, Pebax, PC, PE, |
| | | 23, 24, 27, 36, | 60.0″ to 120.0″ | PU, PP, Aluminum, SS, |
| | | 38, 39, 45, 46, | length × 0.030″ to | Steel, Nylon, Delrin, |
| | | 47, 48, 50 | 0.080″ Main ID, | Rubber |
| | | | 0.010″ × 0.060″ | |
| | | | Next Largest ID × | |
| | | | 0.002″ to 0.020″ | |
| | | | Smallest ID, 0.002″ | |
| | | | to 0.030″ Custom | |
| | | | extruded | |
| | | | Component, off the | |
| | | | shelf component | |
| | Probe | 19, 20, 21, 22, | Custom Shape | Nylon, Pebax, PU, PE, |
| | | 23, 36, 37, 39, | Wall .0025″-.010″ | blends |
| | | 45, 46 | Diameter - 10-30 | |
| | | | French | |
| | Distal Probe Tip | 19, 20, 21, 22, | Custom Extrusion - | Nylon, Pebax, PC, PE, |
| | | 23, 36, 37, 45, | 0.005″ to 0.030″ × | PU, PP |
| | | 46 | 1.0″ to 4.0″ Length | |
| | Catheter Overtube | 19, 20, 21, 22, | ID - .002″-.080″ | Polyimide, PEEK, |
| | | 27, 28, 29, 30, | OD - .006″-.1″ | Nylon, Pebax, ss, steel, |
| | | 31, 32, 33, 36, | L - 4″-125″ | nitinol |
| | | 37, 38, 40, 45, | Custom extruded | |
| | | 46, 47, 48, 49, | Component | |
| | | 50, 51, 52 | | |
| | Catheter Support Wire | 19, 20, 22, 23, | OD - .002″-.080″ | ss, steel, nitinol |
| | | 28, 29, 36, 37, | | |
| | | 39, 40, 45, 46, | | |
| | | 48, 49, 50 | | |
| | Sprayer Tube | 19, 26, 27, 28, | ID - .002″-.080″ | Polyimide, PEEK, |
| | | 29, 34, 35, 36, | OD - .006″-.1″ | Nylon, Pebax, ss, steel, |
| | | 38, 39, 41, 42, | L - 4″-125″ | nitinol |
| | | 43, 44, 45, 47, | Custom extruded | |
| | | 48, 49 | Component | |
| | Distal Cap | 19, 29, 36 | Custom Machined | PC, Ultem, PEEK, PE, |
| | | | or Molded | PU, Delrin |
| | | | Component | |
| | Proximal Cap | 19, 29, 36, 40 | Custom Machined | PC, Ultem, PEEK, PE, |
| | | | or Molded | PU, Delrin |
| | | | Component | |
| | Catheter Support Wire | 20 | OD - .002″-.080″ | ss, steel, nitinol |
| | Pressure Detection | 24 | ID - .002″-.020″ | Polyimide, PEEK, |
| | Tube | | OD - .006″-.03″ | Nylon, Pebax, ss, steel, |
| | | | L - 4″-125″ | nitinol |
| | | | Custom extruded | |
| | | | Component | |
| | Probe Inner Surface | 24, 25 | See probe | See probe |
| | Probe Wall | 24, 25 | See probe | See probe |
| | Delivery Channel | 24, 26, 27, 28, | ID - .002″-.080″ | Polyimide, PEEK, |
| | | 29, 30, 31, 32, | OD - .006″-.1″ | Nylon, Pebax, ss, steel, |
| | | 33, 34, 35, 38, | L - 4″-125″ | nitinol |
| | | 39, 40, 41, 42, | Custom extruded | |
| | | 43, 44, 47, 49, | Component | |
| | | 51, 52 | | |
| | Cryogenic Fluid | 25, 26, 29, 34, | NA | Nitrous oxide, Co2, |
| | | 35, 40, 49 | | helium |

Certain additional features and advantages of the cryogenic catheters, systems, and methods of the present disclosure are provided below.

Translation without Central Rail: In certain embodiments, the entire cryogenic fluid delivery channel assembly of the present disclosure moves longitudinal traversing distal and proximal within the probe. It is possible to rotate this design but it is not required since the sprayers are positioned to achieve a 360-degree spray pattern. No central rail is required. The catheter overtube and catheter support wire provide all the support required to keep the sprayer centrally located within the probe.

Versatile Delivery System: In certain embodiments, the cryogenic catheters of the present disclosure are designed to accommodate both individual and multiple delivery channels. Delivery channels serve to transport cryogenic fluid from the controller to the probe inner surface via the sprayers. The delivery channels are securely housed within the catheter overtube, ensuring stability. The delivery channels do not move internally within the catheter overtube but are secured to each other and/or within the catheter overtube. The delivery channels are cut to identical lengths which facilitates consistent termination at the same radial location within the cryogenic fluid delivery channel assembly.

Cryogenic Fluid Delivery Channel Assembly: In certain embodiments, such as those shown in FIGS. 19-39, the cryogenic catheters and systems of the present disclosure provide flexibility by allowing spraying in the same radial position. In certain other embodiments, such as those shown in FIGS. 40-52, the cryogenic catheters and systems of the present disclosure can include a staggered configuration for sprayers, ensuring each sprayer and its output spray is located and occurs at different longitudinal locations. In certain embodiments of the cryogenic catheters and systems of the present disclosure, all spray patterns can be arranged to uniformly emanate from the center and radiate outward within the probe. In certain embodiments, the cryogenic catheters and systems of the present disclosure can facilitate the use of mesh to improve distribution of the liquid nitrous. In certain embodiments, each sprayer is independently (or multiple delivery channels) activated and begins a treatment by the controller. Once the spray/treatment is activated the controller automatically begin to traverse the cryogenic fluid delivery channel assembly.

The catheters, catheter probes, systems, and methods of using the catheters, catheter probes, and systems for cryogenic ablation of tissue, particularly of the gastrointestinal tract, can further be used in accordance with the devices, systems, methods, and applications of the disclosures of U.S. Pat. Nos. 10,537,387, 10,610,663, and 11,185,367.

### REFERENCES

Citation of a reference herein shall not be construed as an admission that such reference is prior art to the present invention. All references cited herein are hereby incorporated by reference in their entirety. Below is a listing of various references cited with respect to this example:
1. betterhealth.vic.gov.au/health/conditionsandtreatments/diabetes-long-term-effects
2. Liebl A, Mata M, Eschwege E. Evaluation of risk factors for development of complications in Type II diabetes in Europe. Diabetologia 2002; 45: S23-S28.
3. Sjöström L, Lindroos A K, Peltonen M, et al. Lifestyle, diabetes, and cardiovascular risk factors 10 years after bariatric surgery. N Engl J Med 2004; 351:2683-93.
4. Pories W J, Albrecht R J: Etiology of type 2 diabetes mellitus: role of the foregut. World J Surg 25:527-531, 2001
5. Betzel B, Cooiman M I, Aarts E O, Janssen I M C, Wahab P J, Groenen M J M, Drenth J P H, Berends F J. Clinical follow-up on weight loss, glycemic control, and safety aspects of 24 months of duodenal-jejunal bypass liner implantation. Surg Endosc. 2020 January; 34 (1): 209-215.
6. Zervos E E, Agle S C, Warren A J, Lang C G, Fitzgerald T L, Dar M, Rotondo M F, Pories W J. Amelioration of insulin requirement in patients undergoing duodenal bypass for reasons other than obesity implicates foregut factors in the pathophysiology of type II diabetes. J Am Coll Surg. 2010 May; 210 (5): 564-72, 572-4. doi: 10.1016/j.jamcollsurg.2009.12.025. PMID: 20421005.
7 Cherrington A D, Rajagopalan H, Maggs D, Devière J. Hydrothermal Duodenal Mucosal Resurfacing: Role in the Treatment of Metabolic Disease. Gastrointest Endosc Clin N Am. 2017 April; 27 (2): 299-311. doi: 10.1016/j.giec.2016.12.002. PMID: 28292408.
8. van Baar A C G, Holleman F, Crenier L, et al. Endoscopic duodenal mucosal resurfacing for the treatment of type 2 diabetes mellitus: one year results from the first international, open-label, prospective, multicentre study. Gut. 2020; 69 (2): 295-303. doi: 10.1136/gutjnl-2019-318349.
9. ClinicalTrials.gov Identifier-NCT03390322 (clinicaltrials.gov/ct2/show/NCT03390322)
10. Reserved
11. Reserved
12. van Baar A C G, Nieuwdorp M, Holleman F, Soeters M R, Groen A K, Bergman JJGHM. The Duodenum harbors a Broad Untapped Therapeutic Potential. Gastroenterology. 2018 March; 154 (4): 773-777-
13. Dhaliwal_Mo1275 Comparative Assessment of the Structural and Functional Integrity of the Neo-Squamous Epithelium following endoscopic therapy in Barrett's esophagus: A Pilot Study, GIE, POSTER ABSTRACTS| VOLUME 91, ISSUE 6, SUPPLEMENT, AB412, 2020.
14. Erinjeri J P, Clark T W. Cryoablation: mechanism of action and devices. J Vasc Interv Radiol. 2010 August; 21 (8 Suppl): S187-91. doi: 10.1016/j.jvir.2009.12.403. PMID: 20656228; PMCID: PMC6661161.
15. Yakkala C, Chiang C L, Kandalaft L, Denys A, Duran R. Cryoablation and Immunotherapy: An Enthralling Synergy to Confront the Tumors. Front Immunol. 2019 Sep. 24; 10:2283.
16. Erinjeri J P, Clark T W. Cryoablation: mechanism of action and devices. J Vasc Interv Radiol. 2010 August; 21 (8 Suppl): S187-91. doi: 10.1016/j.jvir.2009.12.403.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Citation of a reference herein shall not be construed as an admission that such reference is prior art to the present invention. All references cited herein are hereby incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Although the present invention has been described for the purpose of illustration, it is understood that such detail is solely for that purpose and variations can be made by those skilled in the art without departing from the spirit and scope of the invention which is defined by the following claims.

1. A cryogenic catheter comprising:
a probe at the distal end of the catheter;
a shaft at the proximal end of the catheter; and
a cryogenic fluid delivery channel assembly coupled to the probe and shaft, said cryogenic fluid delivery channel assembly comprising a cryogenic sprayer component, a catheter support wire, and a catheter overtube,
wherein the cryogenic sprayer component comprises at least one sprayer tube and at least one delivery channel, wherein the at least one sprayer tube comprises at least one sprayer through which a cryogenic fluid transported within the at least one delivery channel can be delivered to one or more target areas or treatment zones.

2. The catheter according to claim 1, wherein the sprayer tube is securely fastened between a distal cap and a proximal cap distal cap.

3. The catheter according to claim 2, wherein the cryogenic sprayer component comprises multiple sprayer tubes each containing a single cryogenic fluid delivery channel.

4. The catheter according to claim 2, wherein the cryogenic sprayer component comprises a single sprayer tube containing multiple cryogenic fluid delivery channels.

5. The catheter according to claim 1, wherein the cryogenic sprayer component comprises a single sprayer tube containing one or more cryogenic fluid delivery channels.

6. The catheter according to claim 1, wherein the at least one sprayer comprises multiple sprayers radially aligned, radially staggered, or both radially aligned and staggered in reference to their positions on the sprayer tube.

7. The catheter according to claim 1, wherein the cryogenic fluid delivery channel assembly is configured for translational movement longitudinally between the distal and proximal ends of the catheter.

8. The catheter according to claim 7, wherein the translational movement occurs without requiring a central rail component.

9. The catheter according to claim 1, further comprising a distal probe tip attached to the distal end of the probe.

10. The catheter according to claim 1, wherein the cryogenic fluid delivery channel assembly comprises 1 to 20 delivery channels.

11. The catheter according to claim 1, wherein the cryogenic fluid delivery channel assembly comprises a number of delivery channels selected from the group consisting of 1, 2, 3, 4, 5, 6, 7, and 8 cryogenic fluid delivery channels.

12. The catheter according to claim 1, wherein the sprayers are configured to release the cryogenic fluid either in unison or independently of one another.

13. The catheter according to claim 1, wherein each sprayer is configured to release the cryogenic fluid in a different treatment zone.

14. The catheter according to claim 1, wherein the catheter is configured for selective ablation in the mucosa and submucosa of a gastrointestinal tract of a subject.

15. The catheter according to claim 1, wherein the delivery channels are independently controlled and turned to an on or off position.

16. The catheter according to claim 1, wherein the treatment area or treatment zone comprises mucosal and/or submucosal tissue of the large intestine, small intestine, stomach, esophagus, rectum, and anus.

17. A cryogenic ablation system comprising:
a cryogenic catheter according to claim 1; and
a controller configured to control the functionality of the cryogenic catheter.

18-20. (canceled)

21. The system according to claim 17, wherein each delivery channel is fluidly connected to a delivery channel control valve and a reservoir of cryogenic fluid whereby the delivery channel can be controlled by actuation of each delivery channel control valve.

22-27. (canceled)

28. A method of performing cryogenic ablation of mucosal tissue and/or of both mucosal tissue and submucosal tissue in the gastrointestinal tract of a subject, said method comprising:
(a) providing a cryogenic ablation system according to claim 17;
(b) contacting the cryogenic catheter probe of the system with a target treatment region of the gastrointestinal tract of the subject; and
(c) releasing a cryogenic fluid from the at least one sprayer to treat the target treatment region by cryogenically ablating at least a portion the mucosal tissue or ablating at least a portion of both the mucosal and submucosal tissue of the target treatment region.

29-33. (canceled)

34. A method for performing a medical procedure in a small intestine and/or stomach of a patient in need of said medical procedure, the method comprising:
(a) providing a cryogenic ablation system according to claim 17;
(b) contacting the cryogenic catheter probe of the system with a target treatment region of the small intestine and/or stomach of the patient; and
(c) releasing a cryogenic fluid from the at least one sprayer to treat the target treatment region by cryogenically ablating at least a portion the mucosal tissue or ablating at least a portion of both the mucosal and submucosal tissue of the target treatment region,
thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

35-38. (canceled)
